(19) United States
(12) Patent Application Publication
(10) Pub. No.: US 2023/0047910 A1
(43) Pub. Date: Feb. 16, 2023

(54) SURGICAL FASTENERS, TOOLS, AND METHODS

(71) Applicant: Arthrosurface, Inc., Franklin, MA (US)

(72) Inventors: Nam CHAO, Bedford, MA (US); Steven W. EK, Bolton, MA (US)

(73) Assignee: Arthrosurface, Inc., Franklin, MA (US)

(21) Appl. No.: 17/658,817

(22) Filed: Apr. 11, 2022

Related U.S. Application Data
(60) Provisional application No. 63/173,623, filed on Apr. 12, 2021.
(60) Provisional application No. 63/189,351, filed on May 17, 2021.

(51) Int. Cl. A61F 2/08

(52) U.S. Cl. CPC A61F 2/0811; A61F 2/0805; A61F 2002/0841; A61F 2210/0004; A61F 2220/0016

(57) ABSTRACT

This disclosure includes soft-tissue fasteners for coupling an implant to soft tissue, bone fasteners for coupling an implant to bone, fastener-delivery apparatuses (e.g., tools) for delivering such soft-tissue fasteners and bone fasteners, fastener cartridges for use with certain of the fastener-delivery apparatuses. The present fasteners generally include at least one barbed shaft and an enlarged head spaced from a distal end of the shaft. Some of the present fasteners include two barbed shafts and an enlarged head spanning the two shafts. This disclosure also includes kits that comprise a plurality of fasteners (e.g., a plurality of soft-tissue fasteners, a plurality of bone fasteners, or both). Some of the present kits also include one or more of the present fastener-delivery apparatuses or tools; for example, a plurality of fasteners with a single, reloadable tool; a plurality of tools each pre-loaded with a fastener; and/or a plurality of cartridges each pre-loaded with a fastener and a common tool for use with the cartridges.

### CROSS-REFERENCE TO RELATED APPLICATIONS

This application claims the benefit of priority to U.S. Provisional Patent Application Ser. No. 63/173,623, filed Apr. 12, 2021, and U.S. Provisional Patent Application Ser. No. 63/189,351, filed May 17, 2021, each of which are hereby incorporated by reference in their entirety.

### FIELD OF INVENTION

The present invention relates generally to surgical fasteners, more particularly, but not by way of limitation, to fasteners, tools, and methods for surgical repair of anatomical structures, such as tendons (e.g., the supraspinatus tendon, commonly known as the rotator cuff).

### DESCRIPTION OF RELATED ART

Various surgical fasteners are known, such as for securing an implant, for example for repair of a tear in the supraspinatus tendon (which may be commonly referred to as a rotator cuff tear). For example, United States Patent Application No. US 2008/0188936 discloses certain examples of fasteners and implants for such repairs.

### SUMMARY

This disclosure includes soft-tissue fasteners for coupling an implant to soft tissue, bone fasteners for coupling an implant to bone, fastener-delivery apparatuses (e.g., tools) for delivering such soft-tissue fasteners and bone fasteners, fastener cartridges for use with certain of the fastener-delivery apparatuses. The present fasteners generally include at least one barbed shaft and an enlarged head spaced from a distal end of the shaft. Some of the present fasteners include two barbed shafts and an enlarged head spanning the two shafts. This disclosure also includes kits that comprise a plurality of fasteners (e.g., a plurality of soft-tissue fasteners, a plurality of bone fasteners, or both). Some of the present kits also include one or more of the present fastener-delivery apparatuses or tools; for example, a plurality of fasteners with a single, reloadable tool; a plurality of tools each pre-loaded with a fastener; and/or a plurality of cartridges each pre-loaded with a fastener and a common tool for use with the cartridges.

In some embodiments of the present soft-tissue fasteners for coupling an implant to soft tissue, the soft-tissue fastener comprises: an elongated shaft having a shaft length extending from a shaft proximal end to a shaft distal end, and an internal surface defining an internal passage extending along the shaft length; an enlarged head coupled to the proximal end of the shaft, the enlarged head having a proximal side and a distal side; a plurality of outriggers each extending from the enlarged head in a direction away from the internal passage to an outrigger distal end; and a plurality of barbs each extending along a portion of the shaft length, each of the barbs having a leading edge and trailing edge spaced from the shaft proximal end, the leading edge of each barb being disposed between the trailing edge of that barb and the shaft distal end, where the trailing edge of a second one of the barbs is closer to the shaft proximal end than is the trailing edge of a first one of the barbs; where the shaft, head, outriggers, and barbs are defined by a unitary piece of polymer.

In some embodiments of the present bone fasteners for coupling an implant to bone, the bone fastener comprises: an elongated shaft having a shaft length extending from a shaft proximal end to a shaft distal end, and defining an internal passage extending along the shaft length; an enlarged head coupled to the proximal end of the shaft; a plurality of outriggers extending from the enlarged head in a direction away from the internal passage; and a plurality of first barbs each extending along a portion of the shaft length, each first barb having a leading edge and trailing edge spaced from the proximal end of the shaft, the leading edge of each first barb being disposed between the trailing edge of that first barb and the distal end of the shaft; a plurality of second barbs each extending along a portion of the shaft length, each of the second barbs having a leading edge and a trailing edge disposed between the head and the trailing edges of the first barbs, the leading edge of each second barb being disposed between the trailing edge of that second barb and the distal end of the shaft; where the shaft, head, outriggers, and first and second barbs are defined by a unitary piece of polymer.

In some embodiments of the present apparatuses for delivery of a fastener, the apparatus comprises: an elongated body having a body length extending from a body proximal end to a body distal end; a spike having a spike proximal end coupled to the body distal end, and a spike distal end extending from the body distal end, the spike proximal end having a transverse dimension that is smaller than a transverse dimension of the body distal end such that a shoulder is defined at the spike proximal end, the shoulder configured to abut a head of a fastener received over the spike; a handle coupled to the proximal end of the body; an elongated tubular shield having a shield proximal end and a shield distal end, the shield disposed around the body and movable between a retracted position in which the shield distal end is proximal to the shoulder, and an extended position in which the shield distal end extends past the spike distal end.

In additional embodiments of the present apparatuses for delivery of a fastener, the apparatus comprises: an elongated body having a body length extending from a body proximal end to a body distal end, the elongated body defining internal first and second body passages extending through and between the body proximal end and the body distal end, the first body passage having a central, longitudinal first axis, and the second body passage having a central, longitudinal second axis separated by the first axis by a distance that remains constant along the body length, the first body passage configured to receive a guidewire, and the second body passage configured to receive a trocar or rod; a handle coupled to the proximal end of the body, the handle configured to permit access to the first and second body passages through the body proximal end. In some embodiments, the distal end of the body is configured to be coupled to a proximal end of a fastener cartridge that defines a longitudinal cartridge groove extending through and between the proximal end of the cartridge and a distal end of the cartridge and open to a lateral external surface of the cartridge, and the cartridge further comprises: a spike having a spike proximal end coupled to the distal end of the cartridge, and a spike distal end extending from the distal end of the cartridge, the spike proximal end having a transverse dimension that is smaller than a transverse dimension of the distal end of the cartridge such that a shoulder is defined at the spike proximal end, the shoulder configured to abut a head of a fastener received over the spike. where the proximal end of the cartridge is configured to be removably coupled to the distal end of the body such that the spike is coaxial with the cartridge passage, and the first axis of the first body passage extends through the cartridge groove. In some embodiments, the cartridge further comprises: a rod having a rod proximal end extending proximally from the proximal end of the cartridge, the proximal end of rod configured to be disposed in the second body passage of the body such that the cartridge is movable between a retracted position in which the rod proximal end is disposed in the second body passage, and an extended position in which the rod proximal ends do not extends into the body of the tool; where the rod is coaxial with the spike.

In some embodiments of the present fastener cartridges for use with certain of the present fastener-delivery apparatuses, the fastener cartridge has a proximal end, a distal end, and a lateral external surface between the proximal and distal ends, and the fastener cartridge defines a longitudinal cartridge groove extending through and between the proximal and distal ends and open to the lateral external surface, and the cartridge further comprises: a spike having a spike proximal end coupled to the distal end of the cartridge, and a spike distal end extending from the distal end of the cartridge, the spike proximal end having a transverse dimension that is smaller than a transverse dimension of the distal end of the cartridge such that a shoulder is defined at the spike proximal end, the shoulder configured to abut a head of a fastener received over the spike. where the proximal end of the cartridge is configured to be removably coupled to the distal end of the body such that the spike is coaxial with the cartridge passage, and the first axis of the first body passage extends through the cartridge groove.

In additional embodiments of the present soft-tissue fasteners for coupling an implant to soft tissue, the soft-tissue fastener comprises: an elongated first shaft having a first shaft length extending from a first shaft proximal end to a first shaft distal end, and an internal surface defining a first internal passage extending along the first shaft length; an elongated second shaft having a second shaft length extending from a second shaft proximal end to a second shaft distal end, and an internal surface defining a second internal passage extending along the second shaft length, where the second shaft length is larger than the first shaft length; an enlarged head coupled to the proximal ends of the first and second shafts, the enlarged head having a proximal side and a distal side; a plurality of first barbs each extending along a portion of the first shaft length, each of the first barbs having a leading edge and trailing edge spaced from the first shaft proximal end, the leading edge of each first barb being disposed between the trailing edge of that first barb and the first shaft distal end; a plurality of second barbs each extending along a portion of the second shaft length, each of the second barbs having a leading edge and trailing edge spaced from the second shaft proximal end, the leading edge of each second barb being disposed between the trailing edge of that second barb and the second shaft distal end, where the trailing edge of at least one of the first barbs is closer to the distal side of the head than is the trailing edge of at least one of the second barbs; where the shafts, head, and barbs are defined by a unitary piece of polymer.

In additional embodiments of the present bone fasteners for coupling an implant to bone, the bone fastener comprises: a first elongated shaft, a second elongated shaft, an enlarged head coupled to the proximal ends of the first shaft and the second shaft; and a plurality of barbs coupled to the shafts. In such embodiments, the first elongated shaft has a first shaft length extending from a first shaft proximal end to a first shaft distal end, and defining an internal first passage extending along the first shaft length; and the second elongated shaft has a second shaft length extending from a second shaft proximal end to a second shaft distal end, and defining an internal second passage extending along the second shaft length. Some such embodiments include a plurality of first barbs each extending along a portion of the first shaft length, each first barb having a leading edge and trailing edge spaced from the proximal end of the first shaft, the leading edge of each first barb being disposed between the trailing edge of that first barb and the first shaft distal end; a plurality of second barbs each extending along a portion of the first shaft length, each of the second barbs having a leading edge and a trailing edge disposed between the head and the trailing edges of the first barbs, the leading edge of each second barb being disposed between the trailing edge of that second barb and the first shaft distal end; a plurality of third barbs each extending along a portion of the second shaft length, each third barb having a leading edge and trailing edge spaced from the proximal end of the second shaft, the leading edge of each third barb being disposed between the trailing edge of that third barb and the second shaft distal end; and a plurality of fourth barbs each extending along a portion of the second shaft length, each of the fourth barbs having a leading edge and a trailing edge disposed between the head and the trailing edges of the third barbs, the leading edge of each fourth barb being disposed between the trailing edge of that fourth barb and the second shaft distal end. In some such embodiments, the shafts, head, and barbs are defined by a unitary piece of polymer.

In further embodiments of the present apparatuses for delivery of a fastener, the apparatus comprises: an elongated body having a body length extending from a body proximal end to a body distal end; a pair of spikes each having a spike proximal end coupled to the body distal end, and a spike distal end extending from the body distal end, the spike proximal end having a transverse dimension that is smaller than a transverse dimension of the body distal end such that a shoulder is defined at the spike proximal end, the shoulder configured to abut a head of a fastener received over the spike, where each of the pair of spikes are parallel to and laterally spaced from the other of the pair of spikes; a handle coupled to the proximal end of the body; and an elongated tubular shield having a shield proximal end and a shield distal end, the shield disposed around the body and movable between a retracted position in which the shield distal end is proximal to the shoulder, and an extended position in which the shield distal end extends past the distal ends of the pair of spikes.

In further embodiments of the present apparatuses for delivery of a fastener, the apparatus comprises: an elongated body having a body length extending from a body proximal end to a body distal end, the elongated body defining internal first, second, and third body passages extending through and between the body proximal end and the body distal end, the first body passage having a central, longitudinal first axis, the second body passage having a central, longitudinal second axis separated from the first axis by a distance that remains constant along the body length, the third body passage having a central, longitudinal third axis separated from the second axis by a distance that remains constant along the body length, the first body passage configured to receive a guidewire, the second body passage configured to receive a trocar or rod, and the third body passage configured to receive a trocar or rod; and a handle coupled to the proximal end of the body, the handle configured to permit access to the first, second, and third body passages through the body proximal end. In some such embodiments, the distal end of the body is configured to be coupled to a proximal end of a fastener cartridge that defines a longitudinal cartridge groove extending through and between the proximal end of the cartridge and a distal end of the cartridge and open to a lateral external surface of the cartridge, and the cartridge further comprises: a pair of spikes each having a spike proximal end coupled to the distal end of the cartridge, and a spike distal end extending from the distal end of the cartridge, the spike proximal end having a transverse dimension that is smaller than a transverse dimension of the distal end of the cartridge such that a shoulder is defined at the spike proximal end, the shoulder configured to abut a head of a fastener received over the spike. where the proximal end of the cartridge is configured to be removably coupled to the distal end of the body such that the spike is coaxial with the cartridge passage, and the first axis of the first body passage extends through the cartridge groove. In some such embodiments, the cartridge further comprises: a pair of rods each having a rod proximal end extending proximally from the cartridge proximal end, each rod configured to be disposed in one of the second and third second body passage of the body such that the cartridge is movable between a retracted position in which the rod proximal ends are disposed in respective ones of the second and third body passages, and an extended position in which the rod proximal ends do not extend into the body of the tool; where the pair of rods are each coaxial with one of the pair of spikes.

In further embodiments of the present fastener cartridges for use with certain of the present apparatuses, the fastener cartridge has a proximal end, a distal end, and a lateral external surface between the proximal and distal ends, the fastener cartridge defining a longitudinal cartridge groove extending through and between the proximal and distal ends and open to the lateral external surface, and the cartridge further comprises: a pair of spike having a spike proximal end coupled to the distal end of the cartridge, and a spike distal end extending from the distal end of the cartridge, the spike proximal end having a transverse dimension that is smaller than a corresponding transverse dimension of the distal end of the cartridge such that a shoulder is defined at the spike proximal end, the shoulder configured to abut a head of a fastener received over the spikes; where the proximal end of the cartridge is configured to be removably coupled to the distal end of the body such that the spike is coaxial with the cartridge passage, and the first axis of the first body passage extends through the cartridge groove.

Some embodiments of the present kits comprise; a plurality of soft-tissue fasteners, a plurality of bone fasteners, one of more of the present fastener-delivery apparatuses, and/or one or more of the present cartridges.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1A depicts a perspective view of a first embodiment of the present soft-tissue fasteners.

FIG. 1B depicts a distal end view of the fastener of FIG. 1A.

FIG. 1C depicts a first side view of the fastener of FIG. 1A.

FIG. 1D depicts a second side view of the fastener of FIG. 1A.

FIG. 1E depicts a proximal end view of the fastener of FIG. 1A.

FIGS. 1F-1H depict side cross-sectional views of the fastener of FIG. 1A, along the planes F-F, G-G, and H-H, respectively, of FIG. 1E.

FIG. 2A depicts a perspective view of a first embodiment of the present bone fasteners.

FIG. 2B depicts a distal end view of the fastener of FIG. 2A

FIG. 2C depicts a side view of the fastener of FIG. 2A.

FIG. 2D depicts proximal end view of the fastener of FIG. 2A.

FIG. 2E depicts a side cross-sectional view along the plane D-D of FIG. 2C.

FIG. 2F depicts a side view of a variation of the fastener of FIG. 2A.

FIG. 3A depicts a perspective view of a first embodiment of a tool for delivery of a fastener of FIG. 1A or FIG. 2A, with a fastener of FIG. 2A coupled to a distal end of the tool.

FIG. 3B depicts a side view of the tool of FIG. 3A, shown without the fastener of FIG. 2A.

FIG. 3C depicts a side cross-sectional view of the tool of FIG. 3A, along the plane C-C of FIG. 3B.

FIG. 3D depicts a side view of the tool of FIG. 3A with a shield of the tool omitted.

FIG. 3E depicts the tool of FIG. 3A, with the shield included and disposed in an extended position covering the fastener.

FIG. 4A depicts a perspective view of a second embodiment of a tool for delivery of a fastener of FIG. 1A or FIG. 2A, with a fastener of FIG. 2A coupled to a distal end of the tool.

FIG. 4B depicts a side view of the tool of FIG. 4A, shown without the fastener of FIG. 2A.

FIG. 4C depicts a side cross-sectional view of the tool of FIG. 4A, along the plane C-C of FIG. 4B.

FIG. 4D depicts the tool of FIG. 4A, with the shield disposed in an extended position covering the fastener.

FIG. 5A depicts a perspective view of a third embodiment of a tool for delivery of a fastener of FIG. 1A or FIG. 2A via a cartridge, with a fastener of FIG. 2A coupled to a distal end of the cartridge and the cartridge coupled to a distal end of the tool.

FIG. 5B depicts a side view of the tool of FIG. 5A, shown without the fastener of FIG. 2A.

FIG. 5C depicts a side cross-sectional view of the tool of FIG. 5A, along the plane C-C of FIG. 5B.

FIG. 5D depicts a perspective view of the distal end of the tool, without the cartridge but with a guidewire and a trocar.

FIG. 5E depicts an enlarged side view of a fastener cartridge of the tool of FIG. 5A with a shield of the tool omitted.

FIG. 5F depicts an enlarged lower perspective view of the fastener cartridge of FIG. 5E.

FIG. 5G depicts a cutaway perspective view of the fastener cartridge of FIG. 5E.

FIG. 6A depicts a perspective view of a second embodiment of the present soft-tissue fasteners.

FIG. 6B depicts a side view of the fastener of FIG. 6A.

FIG. 6C depicts a distal end view of the fastener of FIG. 6A.

FIG. 6D depicts a proximal end view of the fastener of FIG. 6A.

FIGS. 6E and 6F depict side cross-sectional views of the fastener of FIG. 6A, along the planes E-E and F-F, respectively, of FIG. 6D.

FIG. 7A depicts a perspective view of an embodiment of a tool for delivery of a fastener of FIG. 6A, with a fastener of FIG. 6A coupled to a distal end of the tool.

FIG. 7B depicts a side view of the tool of FIG. 7A.

FIG. 7C depicts a side cross-sectional view of the tool of FIG. 7A, along the plane C-C of FIG. 7B.

FIG. 7D depicts an additional side view of the tool of FIG. 3A.

FIG. 8A depicts a perspective view of a second embodiment of the present bone fasteners.

FIG. 8B depicts a distal end view of the fastener of FIG. 8A

FIG. 8C depicts a proximal end view of the fastener of FIG. 8A.

FIG. 8D depicts a first side view of the fastener of FIG. 8A.

FIG. 8E depicts a second side view of the fastener of FIG. 8A.

FIG. 9A depicts a perspective view of an embodiment of a tool for delivery of a fastener of FIG. 8A via a cartridge, with a fastener of FIG. 8A coupled to a distal end of the cartridge, and the cartridge coupled to a distal end of the tool.

FIG. 9B depicts a side view of the tool of FIG. 9A.

FIG. 9C depicts a side cross-sectional view of the tool of FIG. 9A, along the plane C-C of FIG. 9B.

FIG. 9D depicts an enlarged first side view of a fastener cartridge of the tool of FIG. 9A.

FIG. 9E depicts an enlarged second side view of the fastener cartridge of the tool of FIG. 9A.

FIG. 9F depicts a distal end view of the tool of FIG. 9A, with the bone fastener coupled to the cartridge.

FIG. 9G depicts a distal end view of the tool of FIG. 9A, with the bone fastener omitted.

FIGS. 10A-10K depict an exemplary sequence of steps for deployment of a fastener of FIG. 8A via the tool of FIG. 9A.

FIG. 11 depicts a perspective view of a number of examples of trays or caddies each configured to hold a plurality of the present fasteners.

FIG. 12A depicts a perspective, cutaway view of the connection of a damaged supraspinatus tendon to the humerus in a human shoulder.

FIG. 12B depicts a perspective, cutaway view of the connection of a damaged supraspinatus tendon to the humerus in a human shoulder, showing a repair in accordance with an example of the present methods.

FIG. 13A depicts a side view of a third embodiment of the present soft-tissue fasteners.

FIG. 13B depicts a side cross-sectional view of the fastener of FIG. 13A, taken along the plane B-B of FIG. 13A.

FIG. 14A depicts a side view of a fourth embodiment of the present soft-tissue fasteners.

FIG. 14B depicts a distal end view of the fastener of FIG. 14A.

FIG. 15A depicts a side view of a fifth embodiment of the present soft-tissue fasteners.

FIG. 15B depicts a distal end view of the fastener of FIG. 15A.

FIG. 16A depicts a side view of a sixth embodiment of the present soft-tissue fasteners.

FIG. 16B depicts a distal end view of the fastener of FIG. 16A.

FIG. 17A depicts a side view of a seventh embodiment of the present soft-tissue fasteners.

FIG. 17B depicts a distal end view of the fastener of FIG. 16A.

FIG. 17C depicts a side cross-sectional view of the fastener of FIG. 19A, taken along the plane C-C of FIG. 17A.

FIG. 18A depicts a side view of an eighth embodiment of the present soft-tissue fasteners.

FIG. 18B depicts a distal end view of the fastener of FIG. 18A.

FIG. 19A depicts a side view of a ninth embodiment of the present soft-tissue fasteners.

FIG. 19B depicts a distal end view of the fastener of FIG. 19A.

FIG. 20 depicts a side view of a tenth embodiment of the present soft-tissue fasteners.

FIG. 21 depicts a side view of an eleventh embodiment of the present soft-tissue fasteners.

FIG. 22 depicts a side view of a twelfth embodiment of the present soft-tissue fasteners.

### DESCRIPTION OF ILLUSTRATIVE EMBODIMENTS

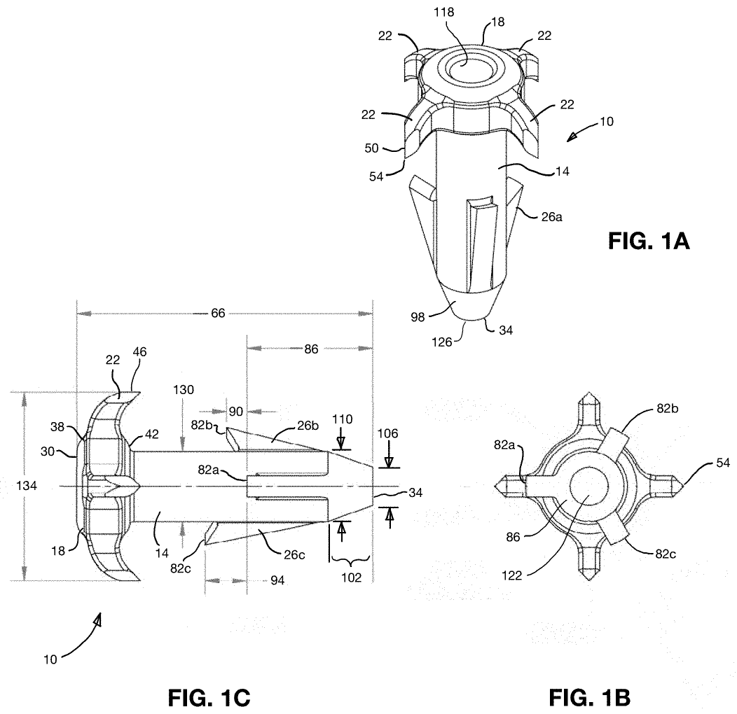

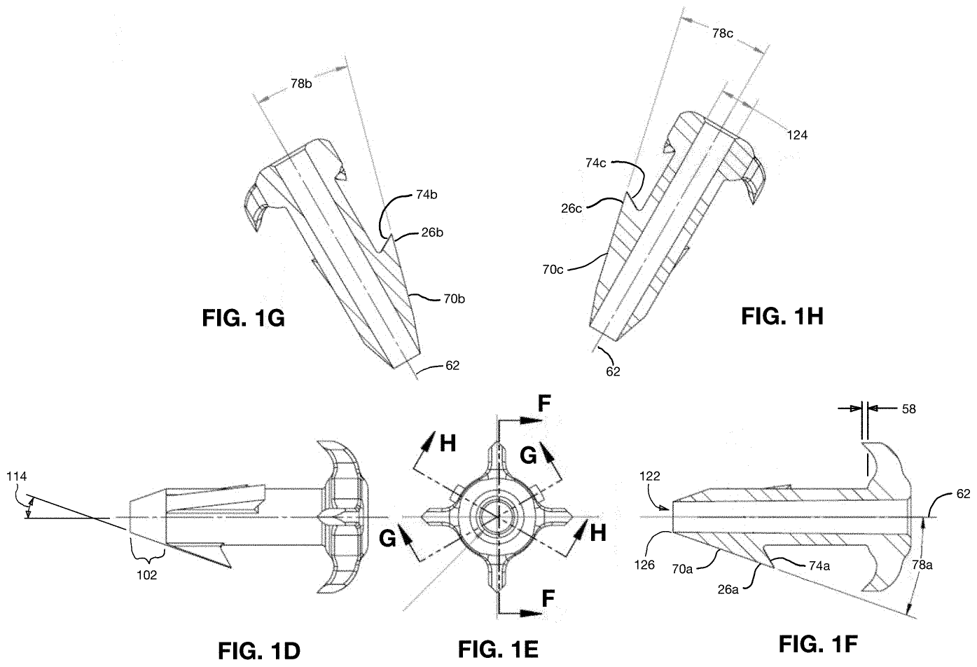

Referring now to the drawings, and more particularly to FIGS. 1A-1H, shown therein and designated by the reference numeral 10 is one embodiment of the present soft-tissue fasteners. In the depicted example, fastener 10 is configured for coupling an implant to soft tissue, such as a tendon (e.g., the supraspinatus tendon or rotator cuff). As shown, fastener 10 comprises an elongated shaft 14, an enlarged head 18, a plurality of outriggers 22, and a plurality of barbs 26a, 26b, 26c.

Shaft 14 extends from a shaft proximal end 30 (and enlarged head 18) to a shaft distal end 34. Shaft distal end 34 is configured to be inserted (along with at least one of barbs 26a, 26b, 26c) into soft tissue of a patient such that the barbs resist removal of the fastener from the soft tissue. For example, in at least some uses, the distal end can first be inserted through an implant such that inserting the distal end into soft tissue couples the implant to the soft tissue. To that end, outriggers 22 each extend outward from head 18 to resist removal of such an implant over the head. Outriggers 22 can be particularly advantageous when the fastener is used in conjunction with a fibrous (e.g., woven) or fabric-like implant, the flexibility of which may otherwise (without outriggers) be more-susceptible to slipping over the head.

As shown, head 18 includes a head proximal side 38, and a head distal side 42. In this configuration, at least a portion of the head distal side is longitudinally aligned with shaft proximal end 30, for example as shown in FIG. 1C.

In some embodiments, such as the one shown, a distal end 46 of each outrigger 22 tapers to an edge 50 and a point 54, which point is configured to assist with engaging an implant that is secured by fastener 10, for example by extending partially into or through the implant (e.g., between fibers of a fibrous implant). To further improve engagement with an underlying implant, outriggers 22 of the depicted embodiment are configured such that the respective distal ends—points 54—extend distally of the distal side (42) of head 18 by a distance 58 (FIG. 1F). Stated another way, when measured parallel to a central, longitudinal axis 62 of the shaft, each of the outrigger distal ends (points 54) is closer to shaft distal end 34 than is at least a portion of distal side 34 of head 18. Other embodiments omit edge 50 but retain point 54 and/or, instead of point 54, may include a rounded, flattened, or roughened distal end (e.g., which may still extend distally of head distal side 42). In the embodiment shown, outriggers 22 are disposed at equiangular intervals around the head. Specifically, four outriggers are disposed at angular intervals of 90 degrees. In other embodiments with a different number of outriggers and equiangular intervals, the equiangular intervals will necessarily also vary (e.g., three outriggers would be disposed at angular intervals of 120 degrees. In other embodiments, outriggers may be disposed at different angular intervals around the head; for example, if one outrigger 22 were omitted from fastener 10, it would leave three outriggers at 90 degree intervals. By way of example, a fastener so modified may be advantageous for use near an edge of an implant (e.g., so the two opposing outriggers separated by an angular interval of 180 degrees could be parallel to the implant edge and the third outrigger could extend inward away from the implant edge).

As shown, each of barbs 26a, 26b, 26c extends longitudinally along a portion of a length 66 of the shaft. In this configuration, each barb extends linearly along the shaft, but in other embodiments, the barbs may be helical or otherwise curved or angled along the shaft. Each of the barbs has a respective distal leading edge 70a, 70b, 70c, and a respective proximal trailing edge 74a, 74b, 74c that is spaced from shaft proximal end 30. For each barb, the leading edge faces distally (toward shaft distal end 34) and is disposed between the shaft distal end and the respective trailing edge. As shown, each of the leading edges is disposed at an acute angle relative to longitudinal axis 62 to facilitate insertion into tissue and/or an implant, and each of the trailing edges is also disposed at an acute angle relative to longitudinal axis 62 to resist removal of the fastener once inserted. In some embodiments, the leading edges of the barbs are disposed at different angles. For example, leading edge 70a of barb 26a is disposed at a first angle 78a, leading edge 70b of barb 26b is disposed at a second angle 78b that is smaller than first angle 78a, and leading edge 70c of barb 26c is disposed at a third angle 78c that is smaller than second angle 78b. These angles (78a, 78b, and 78c) can be selected to manage the force required for insertion and minimize damage to tissue and/or an implant, while maintaining sufficient resistance to removal. For example, for a given barb length, as the angle of the leading edge increases, so does the distance the barb extends from the shaft and the resistance to removal. However, the farther the barb extends outward from the shaft, the greater the chances of tissue fibers being damaged by insertion of the fastener instead of simply permitting the barb to slip past those tissue fibers so they can be engaged by the barb's trailing edge. As such, in the depicted embodiment, the angles of the leading edges decrease with the length of the barbs. In particular, barb 26a has the shortest length (parallel to axis 62) and the greatest leading edge angle 78a, barb 26c has the longest length and the smallest leading edge angle 78c, and barb 26c has a length and a leading edge angle 78b between those of barbs 26a and 26c. In some embodiments, angle 78a is between 15 and 30 degrees, for example, between any two of: 15, 17.5, 20, 22.5, and/or 25 degrees (e.g., between 17.5 and 22.5 degrees); angle 78b is between 10 and 20 degrees, for example, between any two of 10, 12.5, 15, 17.5, and/or 20 degrees (e.g., between 12.5 and 17.5 degrees); and angle 78c is between 7.5 and 17.5 degrees, for example, between any two of 7.5, 10, 12.5, 15, and/or 17.5 degrees (e.g., between 10 and 15 degrees). In the depicted embodiment, angle 78a is between 19 and 20 degrees, angle 78b is between 15 and 16 degrees, and angle 78c is between 12 and 13 degrees.

Additionally, in the depicted embodiment, the trailing edges of the barbs are staggered along the length of shaft to increase the likelihood that at least one of the barbs will securely engage soft tissue. For example, when used with certain tendons (e.g., the supraspinatus tendon), the tendon may have different thicknesses in different regions or may have partial-thickness tears, such that barbs disposed at different distances from the head of the fastener make it more likely that at least one barb will encounter and engage soft tissue of sufficient integrity to resist removal of the fastener. More specifically, in the depicted embodiment, the barbs extend outward from shaft 14 by the same distance and, as a result of their different leading edge angles 78a, 78b, 78c, therefore have different lengths—i.e., trailing edges 74a, 74b, 74c are disposed at different points along the length of the shaft. For example, trailing edge 74b of barb 26b is closer to shaft proximal end 30 than is trailing edge 74a of barb 26a, and trailing edge 74c of barb 26c is closer to shaft proximal end 30 than is trailing edge 74b of barb 26b. As indicated in FIG. 1C, a proximal-most point or edge 82a of barb 26a is a distance 86 from shaft distal end 34, a proximal-most point or edge 82b of barb 26b is a distance 90 proximal of point or edge 82a, and a proximal-most point or edge 82c of barb 26c is a greater distance 94 proximal of point or edge 82a.

In the embodiment shown, barbs 26a, 26b, 26c are disposed at equiangular intervals around the shaft (i.e., around the cross-sectional perimeter of the shaft). Specifically, three barbs are disposed at angular intervals of 120 degrees. In other embodiments with a different number of barb and equiangular intervals, the equiangular intervals will necessarily also vary (e.g., four barbs would be disposed at angular intervals of 90 degrees. In other embodiments, barbs may be disposed at different angular intervals around the shaft; for example, three barbs could be disposed at 90 degree intervals, such that two of the three barbs would be spaced by 180 degrees.

In some embodiments, it can be advantageous for at least one of the barbs to not be radially aligned with any of the outriggers, such that if a barb cuts a slot or creates a tear in an implant when the distal end is inserted through the implant, then at least one outrigger is more likely to not be aligned with that slot or tear and thus reduce the changes of the slot or tear slipping over or around head 18 and outriggers 22. For example, as shown in FIG. 1B, only barb 26a is radially aligned with an outrigger 22, and therefore two of the three barbs (26b, 26c) are not radially aligned with any of the outriggers. Additionally, in some embodiments, the outriggers extend farther outward radially than the barbs, such that—even if all of the barbs cut or tear an implant through which the fastener is inserted—the outriggers (and particularly points 54) are more likely to engage portions of the implant that are outside those cuts or tears.

To further facilitate insertion through an implant and into soft tissue, an outer surface 98 of the shaft defines a tapered section 102 adjacent shaft distal end 34. Tapered section 102 extends from a first outer transverse dimension 106 at shaft distal end 34 and increases in size in a proximal direction to a larger outer transverse dimension 110 that may, as in the depicted embodiment, be substantially equal to an outer transverse dimension along a remainder of the length of the shaft between the tapered section and the head (18). In the embodiment shown, tapered section 102 is tapered linearly at a taper angle 114 relative to axis 62, but in other embodiments may be tapered in a non-linear fashion (e.g., along a curved path). Angle 114 may in some embodiments be equal to angle 78a, and/or may be between 15 and 30 degrees, for example, between any two of: 15, 17.5, 20, 22.5, and/or 25 degrees (e.g., between 17.5 and 22.5 degrees). For example, in the depicted embodiment, angle 114 is between 19 and 20 degrees and is equal to angle 78a such that tapered section 102 and leading edge 70a follow a continuous, linear path.

In the embodiment shown, the shaft (14) is also configured to receive a portion of a delivery tool (described below). In particular, an internal surface 118 of the shaft defines an internal passage 122 that extends through the length of the shaft along axis 62 and through both of proximal end 30 and distal end 34. Passage 122 is configured to receive a portion of a tool (e.g., a spike or trocar) such that that the tool extends through passage 122 and beyond distal end 34 to permit the tool to form a pilot hole in the tissue into which the tool (or a user of the tool) can simultaneously drive the fastener and, after the fastener is inserted into the tissue, the tool can thereafter be removed via the passage (122). In some embodiments, passage 122 has a first inner transverse dimension at the shaft proximal end (30) and a smaller second inner transverse dimension at the shaft distal end (34), such that the passage tapers (e.g., linearly) from smaller at the shaft distal end to larger as the passage approaches the shaft proximal end. For example, in the embodiment shown, passage 122 tapers linearly at an angle 124 of one degree. Such a taper can be advantageous in reducing the resistance to removal of a tool after insertion of the fastener. For example, during insertion of the fastener, soft tissue will be displaced and tend to exert an inward force around the perimeter of a tool in the passage. The provision of a taper, such that a proximal portion of the passage is larger than a distal portion of the passage, typically helps mitigate the compressive forces on a tool in the passage during insertion of the fastener and thereby typically reduces resistance to removal of the tool from the passage after such insertion. In some embodiments, such as the one shown, inner surface 118 and outer surface 98 meet at shaft distal end 34 to form an edge 126. Such an edge at the distal end of the shaft minimizes the cross-sectional area of fastener at the distal end to facilitate insertion of the fastener into tissue.

In the embodiment of FIGS. 1A-1H, shaft 14 has a circular cross-sectional shape, such that an outer transverse dimension 130 is the diameter of a circle that defines the outer perimeter of that shape. In some embodiments, transverse dimension 130 is between any two of: 1.25 mm, 1.35 mm, 1.45 mm, 1.55 mm, 1.65 mm, 1.75 mm, 1.85 mm, 1.95 mm, and/or 2.05 mm (e.g., between 1.55 mm and 1.75 mm). For example, in the embodiment shown, diameter 130 is between 1.6 and 1.7 mm. In other embodiments, the shaft can have other cross-sectional shapes, such as, for example, square, hexagonal, octagonal, or the like.

As described above, the barbs of the fastener extend outwardly from the shaft and therefore span a maximum transverse dimension that is larger than that of the shaft, such as, for example, the barbs extend radially outward to an imaginary circle (perpendicular to axis 62) having a maximum transverse dimension that is between any two of: 130%, 140%, 150%, 160%, 170%, 180%, 190%, 200%, 210%, 220%, and/or 230% of the maximum transverse dimension of the shaft (e.g., between 170% and 190% of the diameter of the shaft). For example, in the embodiment shown, barbs 26a, 26b, 26c each extend radially outward to a point between 1.4 and 1.5 mm from axis 62 (i.e., each of point/edge 82a, 82b, 82c is between 1.4 and 1.5 mm from axis 62).

As also described above, outriggers 22 extend outwardly from enlarged head 82 beyond the lateral extend of the barbs and therefore span a maximum transverse dimension 134 that is larger than that of the barbs, such as, for example, a maximum transverse dimension that is between any two of: 200%, 220%, 240%, 260%, 280%, and/or 300% of the maximum transverse dimension of the shaft (e.g., between 260% and 280% of the maximum transverse dimension of that shaft). For example, in the embodiment shown, outriggers 22 each extend radially outward to a point that is between 2.2 mm and 2.3 mm from axis 62.

In some embodiments, the overall length of the fastener is between 5 mm and 9 mm, such as, for example, between any two of 5 mm, 5.5 mm, 6 mm, 6.5 mm, 7 mm, 7.5 mm, 8 mm, and/or 8.5 mm (e.g., between 5.5 mm and 6.5 mm, between 6.5 mm and 7.5 mm, or between 7.5 mm and 8.5 mm). For example, in the embodiment shown, length 66 is nominally 7 mm, and therefore between 6.5 mm and 7.5 mm. In other embodiments, length 66 can be selected for a particular use. For example, when using a soft-tissue fastener with a tendon that overlies and must move relative to other anatomical structure, it is generally desirable that the distal end of the fastener not extend into the underlying anatomical structures. As an example of one type of tendon, for use with a relatively smaller person with a relatively thinner supraspinatus tendon, a fastener with nominal length of 6 mm may be appropriate; while, for use with a relatively larger person with a relatively thicker supraspinatus tendon, a fastener with a nominal length of 8 mm may be appropriate.

In at least some embodiments, the fastener (i.e., shaft 14, head 18, outriggers 22, and barbs 26a, 26b) are defined by a unitary piece of material. For example, it is sometimes desirable for soft-tissue fasteners to be bioresorbable and, in such instances, the fastener can be molded of a bioresorbable polymer such as poly lactic-co-glycolic acid (PLGA) or polylactic acid (PLA or PLLA). In some uses, it is desirable for complete resorption to occur in less than 9 months (e.g., between 3 months and 9 months).

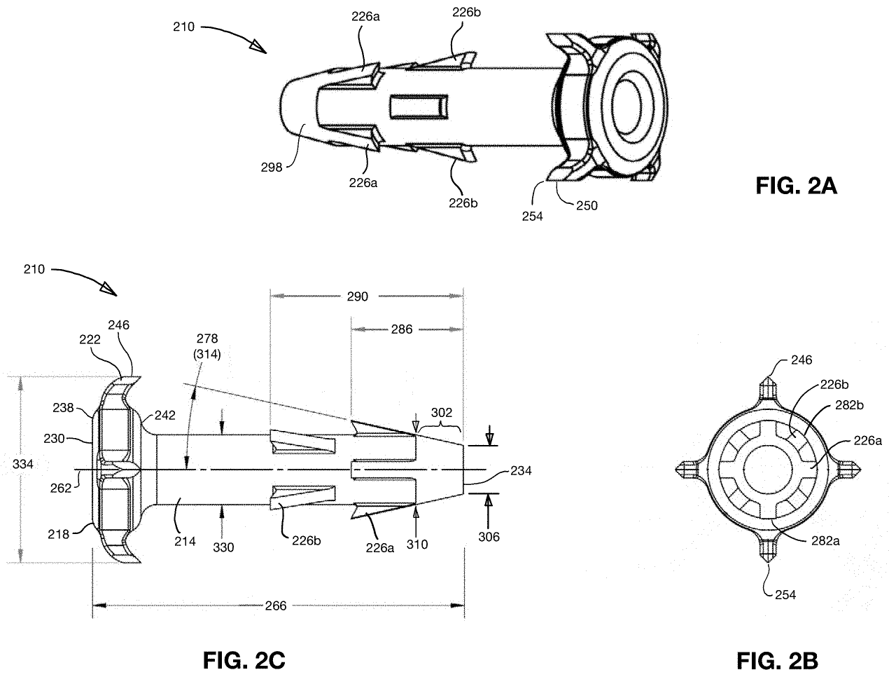

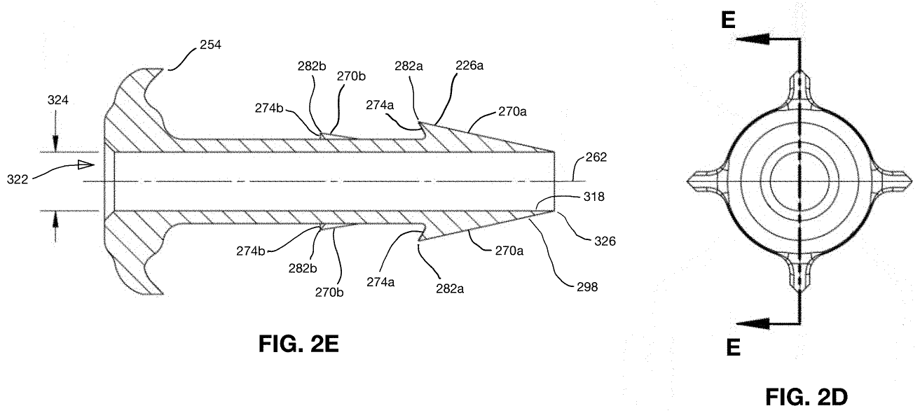

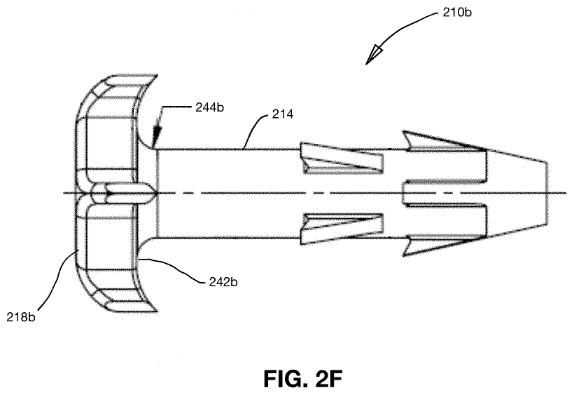

Referring next to FIGS. 2A-2D, a first embodiment of the present bone fasteners 210 is shown. In the depicted example, fastener 210 is configured for coupling an implant to bone, such as the humerus (e.g., to repair the supraspinatus tendon or rotator cuff). As shown, fastener 210 comprises an elongated shaft 214, an enlarged head 218, a plurality of outriggers 222, a plurality of first barbs 226a, and a plurality of second barbs 226b.

Shaft 214 extends from a shaft proximal end 230 (and enlarged head 218) to a shaft distal end 234. Shaft distal end 234 is configured to be inserted (along with at least first barbs 226a) into bone of a patient such that the barbs resist removal of the fastener from the bone. For example, in at least some uses, the distal end can first be inserted through an implant such that inserting the distal end into bone couples the implant to the bone. To that end, outriggers 222 each extend outward from head 218 to resist removal of such an implant over the head. Outriggers 222 can be particularly advantageous when the fastener is used in conjunction with a fibrous (e.g., woven) or fabric-like implant, the flexibility of which may otherwise (without outriggers) be more-susceptible to slipping over the head.

As shown, head 218 includes a head proximal side 238, and a head distal side 242. In this configuration, at least a portion of the head distal side is longitudinally aligned with shaft proximal end 230, for example as shown in FIG. 2C.

In some embodiments, such as the one shown, a distal end 246 of each outrigger 222 tapers to an edge 250 and a point 254, which point is configured to assist with engaging an implant that is secured by fastener 210, for example by extending partially into or through the implant (e.g., between fibers of a fibrous implant). To further improve engagement with an underlying implant, outriggers 222 of the depicted embodiment are configured such that the respective distal ends—points 254—extend distally of another part of the distal side of the respective outrigger. For example, a distal surface of each outrigger is curved to from a concave surface. In this embodiment, points 254 are longitudinally even with distal side 242 of head 218; however, in other embodiments, points 254 may extend distally of the distal side (242) of head 218 by a distance (e.g., similar to outriggers 22 of fastener 10). Other embodiments omit edge 250 but retain point 254 and/or, instead of point 254, may include a rounded, flattened, or roughened distal end (e.g., which may still extend distally of head distal side 242). In the embodiment shown, outriggers 222 are disposed at equiangular intervals around the head. Specifically, four outriggers are disposed at angular intervals of 90 degrees. In other embodiments with a different number of outriggers and equiangular intervals, the equiangular intervals will necessarily also vary (e.g., three outriggers would be disposed at angular intervals of 120 degrees. In other embodiments, outriggers may be disposed at different angular intervals around the head; for example, if one outrigger 222 were omitted from fastener 210, it would leave three outriggers at 90 degree intervals. By way of example, a fastener so modified may be advantageous for use near an edge of an implant (e.g., so the two opposing outriggers separated by an angular interval of 180 degrees could be parallel to the implant edge and the third outrigger could extend inward away from the implant edge).

As shown, each of the first and second barbs (226a, 226b) extends longitudinally along a portion of a length 266 of the shaft. In this configuration, each barb extends linearly along the shaft, but in other embodiments, the barbs may be helical or otherwise curved or angled along the shaft. Each of the barbs has a respective distal leading edge 270a, 270b, and a respective proximal trailing edge 274a, 274b that is spaced from shaft proximal end 230. For each barb, the leading edge faces distally (toward shaft distal end 234) and is disposed between the shaft distal end and the respective trailing edge. As shown, each of the leading edges is disposed at an acute angle relative to longitudinal axis 262 to facilitate insertion into tissue, and each of the trailing edges is also disposed at an acute angle relative to longitudinal axis 262 to resist removal of the fastener once inserted.

In the embodiment shown, the leading edges of the first and second barbs are all disposed at a common angle 278. The angle or angles of the leading edges of the first and second barbs can be selected to manage the force required for insertion and minimize damage to bone tissue, while maintaining sufficient resistance to removal. For example, for a given barb length, as the angle of the leading edge increases, so does the distance the barb extends from the shaft and the resistance to removal. However, the farther the barb extends outward from the shaft, the greater the chances of tissue fibers being damaged by insertion of the fastener instead of simply permitting the barb to slip past those tissue fibers so they can be engaged by the barb's trailing edge. In some embodiments, angle 278 is between 7.5 and 17.5 degrees, for example, between any two of 7.5, 10, 12.5, 15, and/or 17.5 degrees (e.g., between 10 and 15 degrees). In the depicted embodiment, angle 278 is between 12 and 13 degrees. In other embodiments, the leading edges of the first barbs are disposed at a first angle, and the leading edges of the second barbs are disposed at a second angle that is different than the first angle. For example, the leading edges of first barbs 226a can be disposed at first angle that is between 7.5 and 17.5 degrees, for example, between any two of 7.5, 10, 12.5, 15, and/or 17.5 degrees (e.g., between 10 and 15 degrees, or between 12 and 13 degrees), and the leading edges of the second barbs 226b can be disposed at a second angle that is between 10 and 20 degrees, for example, between any two of 10, 12.5, 15, 17.5, and/or 20 degrees (e.g., between 12.5 and 17.5 degrees, or between 15 and 16 degrees).

Additionally, in the depicted embodiment, the trailing edges of the first barbs (226a) and the trailing edges of the second barbs (226b) are staggered along the length of shaft to increase the likelihood that at least one of the barbs will securely engage bone tissue. For example, when used to secure an implant over a portion of a bone (e.g., the humerus), the implant may have different thicknesses in different regions or may have not lay perfectly against the bone, such that barbs disposed at different distances from the head of the fastener make it more likely that at least one set of barbs will encounter and engage bone tissue underlying the implant to resist removal of the fastener. More specifically, in the depicted embodiment, first barbs 226a are disposed closer to shaft distal end 234 than to shaft proximal end 230, and second barbs 226b are disposed closer to the shaft proximal end 230 such that their trailing edges 274b are disposed between the trailing edges 274a of the first barbs and shaft and head 218. Stated another way, trailing edges 274b of second barbs 226b are closer to shaft proximal end 230 than are trailing edges 274a of first barbs 226a. As indicated in FIG. 2C, a proximal-most points or edges 282a of first barbs 226a are a distance 286 from shaft distal end 234, and proximal-most points or edges 282b of second barbs 226b are a greater distance 290 proximal of points or edges 282a.

In the embodiment shown, first barbs 226a are disposed at equiangular intervals around the shaft (i.e., around the cross-sectional perimeter of the shaft), and second barbs 226b are also disposed at equiangular intervals around the shaft. Specifically, four first barbs 226a are disposed at angular intervals of 90 degrees, and four second barbs 226b are disposed at angular intervals of 90 degrees. In other embodiments with a different number of barb and equiangular intervals, the equiangular intervals will necessarily also vary (e.g., three barbs would be disposed at angular intervals of 120 degrees. In other embodiments, barbs may be disposed at different angular intervals around the shaft; for example, three barbs could be disposed at 90 degree intervals, such that two of the three barbs would be spaced by 180 degrees.

In some embodiments, it can be advantageous for at least the first barbs or the second barbs to not be radially aligned with any of the outriggers, such that if a barb cuts a slot or creates a tear in an implant when the distal end is inserted through the implant, then the outriggers are more likely to not be aligned with that slot or tear and thus reduce the changes of the slot or tear slipping over or around head 218 and outriggers 222. For example, as shown in FIG. 2B, only first barbs 226a are radially aligned with outriggers 222, and second barbs 26b are not radially aligned with any of the outriggers. Additionally, in some embodiments, the outriggers extend farther outward radially than the barbs, such that—even if all of the barbs cut or tear an implant through which the fastener is inserted—the outriggers (and particularly points 254) are more likely to engage portions of the implant that are outside those cuts or tears. In other embodiments, all of the first and second barbs can be rotated relative to the outriggers such that none of the barbs are radially aligned with the outriggers.

To further facilitate insertion through an implant and into soft tissue, an outer surface 298 of the shaft defines a tapered section 302 adjacent shaft distal end 234. Tapered section 302 extends from a first outer transverse dimension 306 at shaft distal end 234 and increases in size in a proximal direction to a larger outer transverse dimension 310 that may, as in the depicted embodiment, be substantially equal to an outer transverse dimension along a remainder of the length of the shaft between the tapered section and the head (218). In the embodiment shown, tapered section 302 is tapered linearly at a taper angle 314 relative to axis 262, but in other embodiments may be tapered in a non-linear fashion (e.g., along a curved path). Angle 314 may in some embodiments be equal to angle 278, and/or may be between 7.5 and 17.5 degrees, for example, between any two of 7.5, 10, 12.5, 15, and/or 17.5 degrees (e.g., between 10 and 15 degrees, or between 12 and 13 degrees). For example, in the depicted embodiment, angle 314 is between 19 and 20 degrees and is equal to angle 278 such that tapered section 302 and leading edge 270 follow a continuous, linear path.

In the embodiment shown, the shaft (214) is also configured to receive a portion of a delivery tool (described below). In particular, an internal surface 318 of the shaft defines an internal passage 322 that extends through the length of the shaft along axis 262 and through both of proximal end 230 and distal end 234. Passage 322 is configured to receive a portion of a tool (e.g., a spike or trocar) such that that the tool extends through passage 322 and beyond distal end 234 to permit the tool to form a pilot hole in the tissue into which the tool (or a user of the tool) can simultaneously drive the fastener and, after the fastener is inserted into the tissue, the tool can thereafter be removed via the passage (322). In some embodiments, passage 322 has a first inner transverse dimension at the shaft proximal end (230) and a smaller second inner transverse dimension at the shaft distal end (234), such that the passage tapers (e.g., linearly) from smaller at the shaft distal end to larger as the passage approaches the shaft proximal end. For example, in the embodiment shown, passage 322 tapers linearly at an angle 324 of one degree. Such a taper can be advantageous in reducing the resistance to removal of a tool after insertion of the fastener. For example, during insertion of the fastener, soft tissue will be displaced and tend to exert an inward force around the perimeter of a tool in the passage. The provision of a taper, such that a proximal portion of the passage is larger than a distal portion of the passage, typically helps mitigate the compressive forces on a tool in the passage during insertion of the fastener and thereby typically reduces resistance to removal of the tool from the passage after such insertion. In some embodiments, such as the one shown, inner surface 318 and outer surface 298 meet at shaft distal end 234 to form an edge 326. Such an edge at the distal end of the shaft minimizes the cross-sectional area of fastener at the distal end to facilitate insertion of the fastener into tissue.

In the embodiment of FIGS. 2A-2E, shaft 214 has a circular cross-sectional shape, such that an outer transverse dimension 330 is the diameter of a circle that defines the outer perimeter of that shape. In some embodiments, transverse dimension 330 is between 1.75 mm and 2.5 mm, for example, between any two of: 1.75 mm, 1.85 mm, 1.95 mm, 2.05 mm, 2.15 mm, 2.25 mm, 2.35 mm, 2.45 mm, and/or 2.5 mm (e.g., between 2.05 mm and 2.25 mm). For example, in the embodiment shown, diameter 330 is between 2.1 and 2.2 mm. In other embodiments, the shaft can have other cross-sectional shapes, such as, for example, square, hexagonal, octagonal, or the like.

As described above, the barbs of the fastener extend outwardly from the shaft and therefore span a maximum transverse dimension that is larger than that of the shaft, such as, for example, the barbs extend radially to an imaginary circle (perpendicular to axis 62) having a maximum transverse dimension that is between any two of: 110%, 120%, 130%, 140%, 150%, 160%, 170%, 180%, 190%, and/or 200% of the maximum transverse dimension of the shaft (e.g., between 130% and 140% of the diameter of the shaft). For example, in the embodiment shown, barbs 226a, 226b each extend radially outward to a point between 1.4 and 1.5 mm from axis 262 (i.e., each of point/edge 282a, 282b is between 1.4 and 1.5 mm from axis 262).

As also described above, outriggers 222 extend outwardly from enlarged head 282 beyond the lateral extend of the barbs and therefore span a maximum transverse dimension 334 that is larger than that of the barbs, such as, for example, a maximum transverse dimension that is between any two of: 220%, 230%, 240%, 250%, 260%, 270%, 280%, 290%, 300%, 310%, and/or 320% of the maximum transverse dimension of the shaft (e.g., between 260% and 270% of the maximum transverse dimension of that shaft). For example, in the embodiment shown, outriggers 222 each extend radially outward to a point that is between 2.8 mm and 2.9 mm from axis 262.

In some embodiments, the overall length of the fastener is between 9 mm and 15 mm, such as, for example, between any two of 9 mm, 10 mm, 11 mm, 12 mm, 13 mm, 14 mm, and/or 15 mm (e.g., between 10 mm and 13 mm). For example, in the embodiment shown, length 266 is nominally 11.5 mm, and therefore between 11 mm and 12 mm. In other embodiments, length 266 can be selected for a particular use. For example, when using a bone fastener with an implant through which the bone fastener extends, it is generally desirable that the fastener have a shaft length sufficient to permit the fastener to extend through the implant and into the bone a sufficient distance for the trailing edges of all of the barbs to engage bone.

In at least some embodiments, the fastener (i.e., shaft 214, head 218, outriggers 222, and barbs 226a, 226b) are defined by a unitary piece of material. For example, it is sometimes desirable for bone fasteners to be durable and non-bioresorbable and, in such instances, the fastener can be molded of a non-bioresorbable polymer such as polyether ether ketone (PEEK).

FIG. 2F depicts a side view of a variation 210b of the fastener of FIG. 2A. Fastener 210b is substantially similar to fastener 210a with the primary difference being that distal side 42b of head 18b is entirely concave and forms a continuous concave surface with the distal side of outriggers 22, as shown. Specifically, in the depicted embodiment, a radius 44b forms a concave surface that meets the nominal diameter of shaft 214.

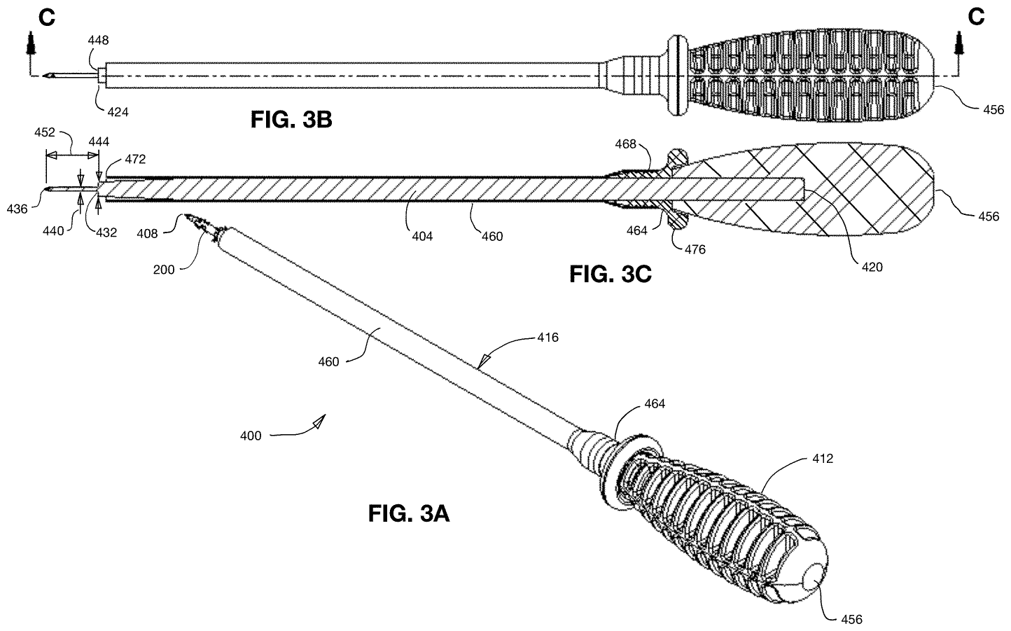

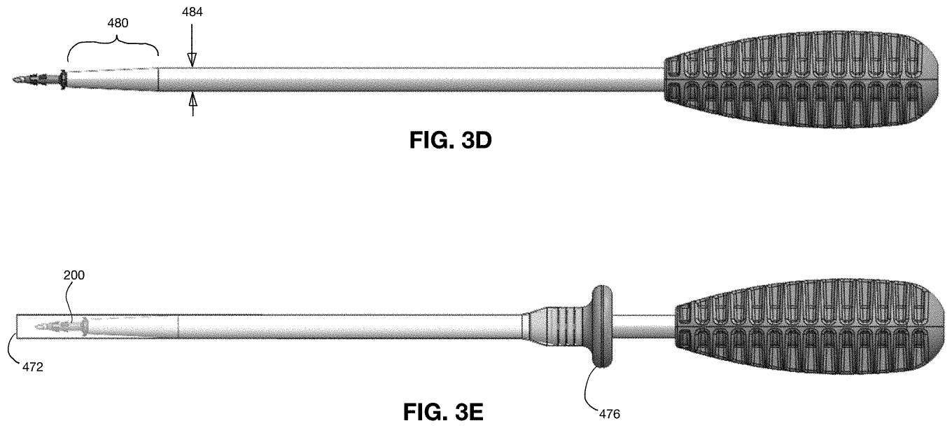

Referring now to FIGS. 3A-3E, a first embodiment of a tool 400 for delivery of a fastener 10 (FIGS. 1A-1H) or fastener 210 (FIGS. 2A-2E), shown with a fastener 210 coupled to a distal end of the tool. In the embodiment shown, tool 400 comprises an elongated body 404, a spike 408, and a handle 412, and a movable a shield assembly 416. Body 404 has a proximal end 420 and a distal end 424. Spike 408 has a spike proximal end 432 coupled to body distal end 424, and a spike distal end 436 extending from the body distal end. In the embodiment shown, spike 408 is unitary (formed of a single piece of material) with body 404. For example, body 404 and spike 408 may be machined from a single piece of material (e.g., stainless steel). As shown, spike 408 is sized to extend through the central passage (e.g., 122, 322) of a fastener that the tool is configured to deliver. For example, for a fastener with a passage having a minimum internal diameter of 1 mm, the spike may have an outer diameter of 0.90 mm or 0.95 mm. Additionally, the interface between spike 408 and body 404 is configured to allow a user to push or drive the fastener into tissue (soft tissue or bone). For example, as shown in FIG. 3C, spike proximal end 432 has a transverse dimension 440 that is smaller than a transverse dimension 444 of the body distal end, such that a shoulder 448 is defined at the spike proximal end. Shoulder 448 configured to abut a head (e.g., 18, 218) of a fastener received over the spike, such as is illustrated for fastener 210.

Spike 408 has a length 452 that is greater than a length (e.g., 66, 266) of a fastener with which tool 400 is configured to be used, such that distal end 436 extends beyond the fastener distal end to allow spike distal end 436 to create a pilot hole in tissue (soft tissue or bone tissue) into which the fastener can follow. In some embodiments, spike length 452 is between 3 mm and 8 mm longer than the length (e.g., 66, 266) of a fastener intended for use with the tool, such as, for example, between any two of: 3 mm, 4 mm, 5 mm, 6 mm, 7 mm, 8 mm, and/or 9 mm (e.g., between 4 mm and 6 mm) longer than the corresponding fastener length. For example, in the depicted embodiment, tool 400 is configured to deliver bone fasteners 210 with an overall length of 11.5 mm, and spike 408 is configured to extend 5 mm beyond fastener distal end, such that spike 408 has a length 452 of 16.5 mm (e.g., between 16 mm and 17 mm). In other embodiments, such as those configured solely for delivery of soft-tissue fasteners (e.g., 10) having a length of 6, 7, and/or 8 mm, spike length 452 may be 5 mm longer than the a single intended fastener length (e.g., 11, 12, or 13 mm) for use with a single intended fastener configuration, or may be 5 mm longer than the longest intended fastener length (e.g., 13 mm) for use with any of several intended fastener lengths.

In the embodiment shown, which is configured to deliver a bone fastener, spike distal end 436 is configured as a conventional trocar tip, in which three or four planar surfaces converge at the spike distal end to form a point or chisel tip. In testing of the present fasteners, trocar tips have demonstrated particularly effective performance in forming an initial pilot hole into which the tool (via shoulder 444) can drive bone fastener 210 (e.g., without separately drilling a pilot hole prior to insertion of the spike distal end 436 and fastener 210 together). In other embodiments of tool 400, such as those configured solely for delivery of soft-tissue fasteners (e.g., 10), spike distal end 436 can be configured with a conical point.

In the depicted embodiment, handle 412 is coupled to body proximal end 420. In this configuration, handle 412 is generally symmetrical around a longitudinal axis so that use is not dependent on rotational position. Handle 412 is coupled to body 404 with sufficient rigidity to permit a user to strike (e.g., with a mallet) a proximal end 456 of handle 412 to drive a bone fastener (e.g., 210) into bone. Handle can be machined or molded from a material sufficiently rigid to receive such a strike and that is capable of sterilization, such as, for example, metals and metal alloys such as aluminum or stainless steel, or any of various polymers such as polyphenylsulfone (PPSU).

Shield assembly 416 comprises an elongated tubular shield 460 coupled in fixed relation to a hub 464. Shield 460 has a proximal end 468 coupled to hub 464 and a distal end 472 extending from hub 464. Hub 464 also defines a flange 476 configured to be engaged by a user to retract the shield assembly. More particularly, shield assembly 416 is disposed around body 404 and movable (e.g., slideable) between a retracted position (FIGS. 3A, 3B) in which shield distal end 472 is proximal to shoulder 448, and an extended position (FIG. 3E) in which shield distal end 472 extends past (distal of) spike distal end 436. In use, such as during arthroscopic surgery, the shield can be disposed in the extended position when the fastener is first passed through a cannula or port to a surgical site, such as to protect the fastener and prevent tissue from engaging or “catching” on the fastener before the fastener reaches a desired insertion position. Once the spike distal end 436 and fastener are disposed near a desired insertion position, a user can engage flange 476 to pull shield assembly 416 proximally relative to body 404 and handle 412 (e.g., from the extended position of FIG. 3A to the retracted position of FIG. 3E). A user can, simultaneously or subsequently, press or strike handle 408 in a distal direction to drive the spike distal end (436) and fastener (e.g., 210) into a desired location in soft tissue or bone tissue.

To facilitate insertion of a fastener and the ability of the shield to move relative to the body and fastener, the depicted configuration of body 404 includes a tapered section 480 at shoulder 448. Specifically, tapered section 480 tapers from a nominal transverse dimension (diameter) 484 to the relatively smaller transverse dimension (diameter) 444 at shoulder 448. In this configuration, dimension 444 is larger than the corresponding maximum transverse dimension of the fastener shaft (e.g., 130, 330) but smaller than the corresponding maximum transverse dimension of the outriggers (e.g., 134, 334), such that the outriggers are permitted a degree of flexibility in the longitudinal direction during insertion (e.g., to account for variations in underlying surfaces and facilitate full insertion of the fastener shaft and barbs). Of course, to facilitate the described movement of the shield relative to body 404 and a fastener disposed thereon, shield 460 has an inner transverse dimension (e.g., diameter) that is larger than corresponding maximum transverse dimensions of the fastener (e.g., dimension 134 or 334 of the outriggers) and of the body 404. To facilitate user visibility during use, shield 460 is transparent in at least some embodiments to enable a user to perceive the position of the fastener and spike distal end relative to the distal end of the shield. For example, shield 460 may be formed of polycarbonate or other suitably durable polymers that are capable of being sterilized and/or are transparent or sufficiently translucent to permit a user to perceive the position of a fastener within the shield.

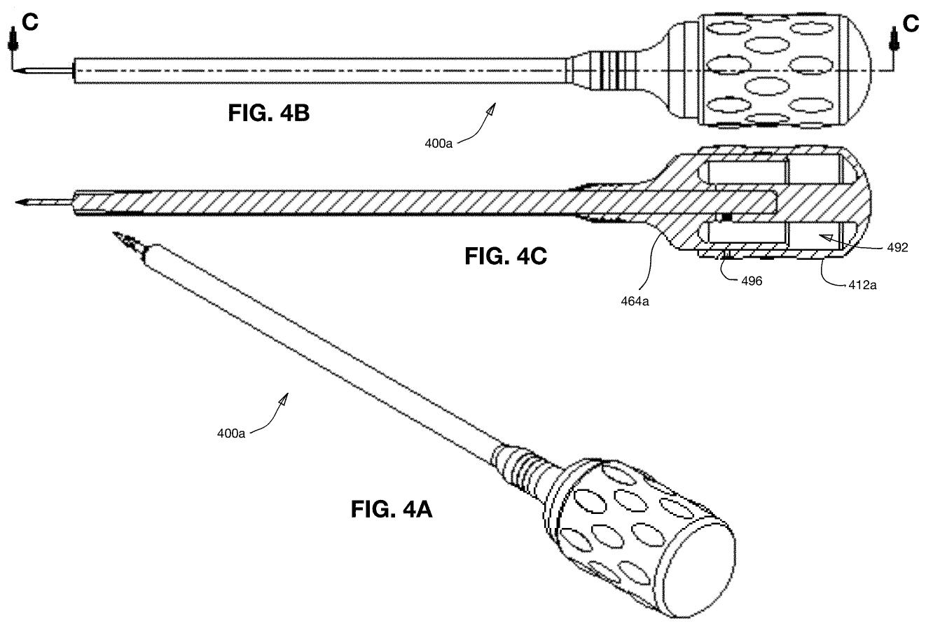

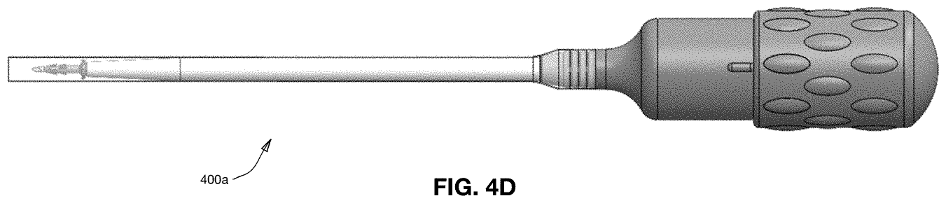

FIGS. 4A-4D depict a second embodiment 400a of a tool for delivery of a fastener 10 (FIGS. 1A-1H) or fastener 210 (FIGS. 2A-2E), shown with a fastener 210 coupled to a distal end of the tool. Tool 400a is substantially similar to tool 400, with the primary difference being that tool 400a includes a handle 412a that is shaped to interact with an elongated shield hub 464a. More particularly, handle 412a defines an annular recess 488 facing distally and configured to receive a proximal portion 492 of hub 464a. In the depicted embodiment, tool 400a is particularly well-suited for implementations in which the tool is provided to a user with the tool pre-loaded with a corresponding fastener (e.g., 210). For example, the longitudinal overlap of hub 464a and handle 412a facilitates the inclusion of a frangible pin 496 (shown broken in FIG. 4C) that extends through the handle and into or proximal to the hub when the shield is in the extended position (FIG. 4D) to resist movement of the shield until a user applies sufficient longitudinal compressive force between the handle and the hub to break the pin, and thereby retract the shield and expose the fastener. In other embodiments, instead of the frangible pin, a detent or other structure can be used that resists inadvertent retraction of the shield but still permits a user to overcome that resistance by hand when ready to deploy the fastener. With tools in a pre-loaded state, the present kits can comprise a plurality of (e.g., three to five) bone tools (e.g., 400a) pre-loaded with bone fasteners (e.g., 210) and having their respective shield assemblies in an extended position, and/or a plurality of (e.g., six to eight) soft-tissue tools (e.g., 400a) pre-loaded with soft-tissue fasteners (e.g., 10) and having their respective shield assemblies in an extended position. Such a kit can be sterile and enclosed in a sealed package (e.g., sealed tray or a blister pack).

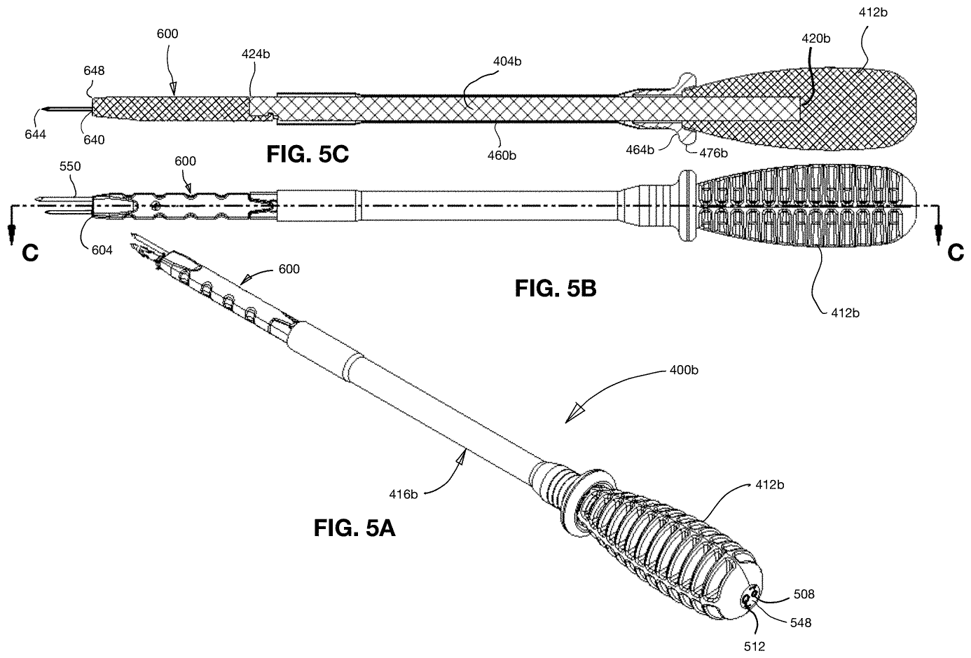

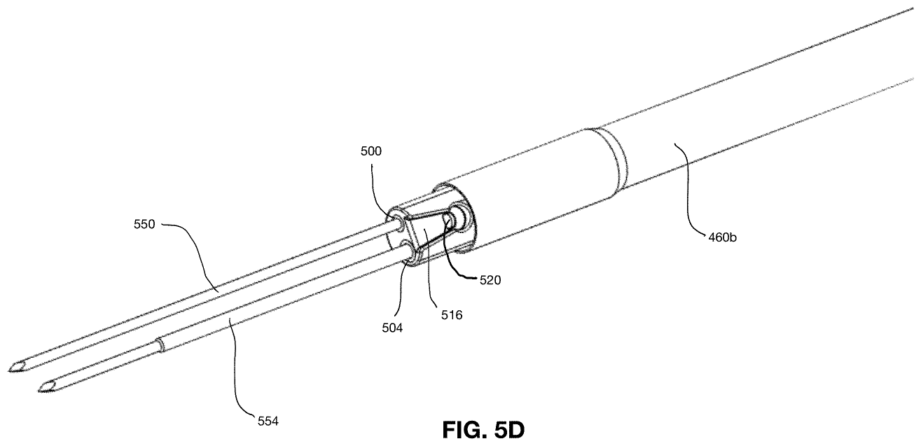

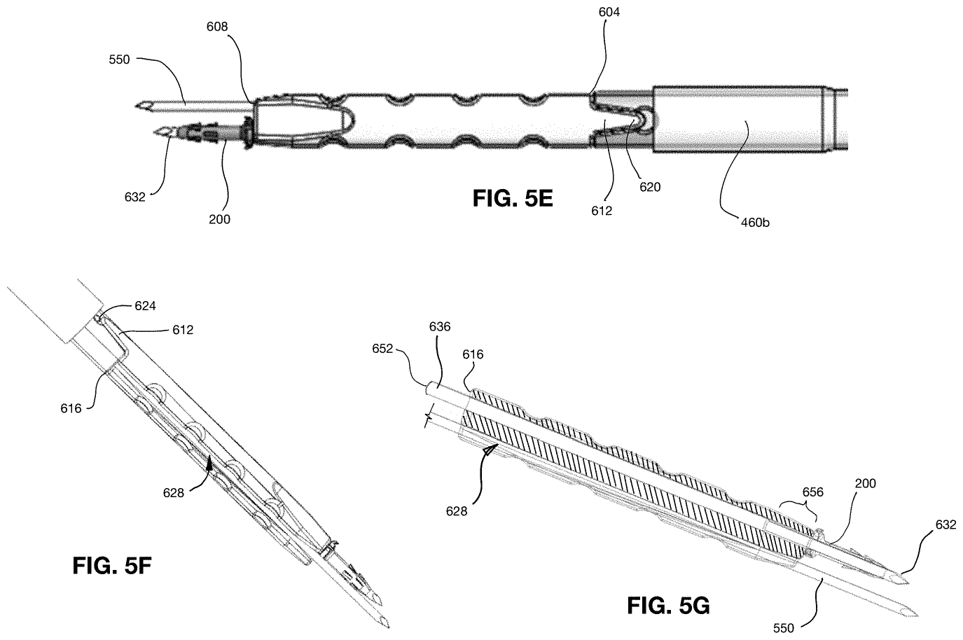

Referring now to FIGS. 5A-5F, FIGS. 5A-5C depict a third embodiment of a tool 400b including a cartridge 600 for delivery of a bone fastener of FIG. 2A with a bone fastener of FIG. 2A coupled to a distal end of the cartridge; FIG. 5D depicts tool 400b without the cartridge but with a guidewire and a trocar; and FIGS. 5E-5G show enlarged views of the cartridge. Tool 400b is similar in several respects to tool 400, with the primary differences being that tool 400b is configured to facilitate pre-drilling or pre-formation of pilot holes in bone with a spatial reference provided by a guidewire, after which a fastener cartridge 600 can be slipped laterally over the guidewire and engaged by a distal end of the tool to align the fastener with, and insert the fastener into, the pilot hole.

More particularly, body 404b of tool 400b extends from a body proximal end 420b to a body distal end 424b. In this configuration, body 404b defines internally a first body passage 500 and a second body passage 504, both of which extend through and between body proximal end 420 and body distal end 424. First body passage 500 has a central, longitudinal first axis, and second body passage 504 has a central, longitudinal second axis separated from the first axis by a distance that remains constant along the body length (i.e., such that the first axis (of the first body passage) is parallel to the second axis (of the second body passage). In the embodiment shown, first body passage 500 is configured to receive a guidewire (e.g., 550), and second body passage 504 configured to receive a trocar (e.g., 554) or rod (e.g., 636). The trocar or rod may be larger than the guidewire, and therefore second body passage 504 may be larger than first body passage 500. Handle 412b is similar to handle 412, with the primary exception that handle 412b is configured to permit access to the first and second body passages 500, 504 through the body proximal end (i.e., through a portion of the handle). More particularly, in this configuration, the handle defines corresponding first and second passages 508, 512 that correspond to (and align with) ones of the first and second passages 500, 504 of the body such that first body passage 500 and first handle passage 508 cooperate to define an essentially continuous first passage between and through handle proximal side 456 and body distal end 424, and such that second body passage 504 and second handle passage 512 cooperate to define an essentially continuous second passage between and through handle proximal side 456 and body distal end 424.

In the embodiment shown, body distal end 424b is configured to engage a proximal end (604) of the cartridge. More particularly, an outer surface of body 404b defines a recess 516 that extends longitudinally inward from body distal end 424b and radially inward from outermost portions of the cross-sectional perimeter of the body. Recess 516 narrows at it extends proximally to facilitate rotational alignment of the cartridge relative to body 404b, and recess 516 further includes a radially deeper portion 520 at a proximal portion of the recess to receive a detent or portion of a projection (612) of the cartridge, as described below, to resist inadvertent longitudinal separation of the cartridge from body distal end 424.

In the depicted embodiment, cartridge 600 includes a proximal end 604 and a distal end 608. Proximal end 604 is configured to engage body distal end 424, and distal end 608 is configured to receive a bone fastener (e.g., 210). Proximal end 604 is configured to engage body distal end 424 via a projection 612 that extends longitudinally outward from a longitudinal engagement surface 616. As shown, projection 612 narrows to a proximal end 620 in a way that corresponds to that of recess 516 to ensure radial alignment of the cartridge relative to body 404 as the cartridge and tool body are pushed together. Projection 612 also includes a radial projection or detent 624 that is configured to extend into the radially deeper portion 520 to resist separation of the cartridge from body distal end 424.

Cartridge 600 defines a longitudinally extending cartridge groove 628 that extends through and between proximal end 604 and distal end 608 of the cartridge, and is open to a lateral external surface of the cartridge such that the cartridge can be laterally slipped over guidewire 550 (such that guidewire 550 is received in groove 628) while a first end of the guidewire is disposed in tissue and a second end of the guidewire extends into first body passage 500 of the tool. Additionally, cartridge 600 comprises a spike 632 coupled to and extending distally from distal end 608 of the cartridge, and a rod 636 coupled to and extending proximally from proximal end 604 of the cartridge. Spike 632 has a spike proximal end 640 coupled to the distal end 608 of the cartridge, and a spike distal end 644 extending from distal end 608 of the cartridge. As described above for spike 408 of tool 400, proximal end 640 of spike 632 has a transverse dimension that is smaller than a transverse dimension of a corresponding portion of distal end 608 of the cartridge, such that a shoulder 648 is defined at spike proximal end 640. And, as with shoulder 448 of tool 400, shoulder 648 is configured to abut a head of a fastener received over the spike (632). Rod 636 has a proximal end 652 extending from cartridge proximal end 608, as shown. In this configuration, the cartridge is configured such that when guidewire 550 is fully received in cartridge groove 628, rod 636 is coaxial with first body passage 504, and cartridge can therefore be moved toward body distal end 424 to insert rod 636 into first body passage 504 and seat cartridge projection 612 into recess 516 to engage tool body 400. Once seated in the respective structures of tool body 400, guidewire 550, rod 636, and projection 612 cooperate to resist movement of cartridge 600 relative to tool body 400. In some embodiments, such as the one shown, spike 632 and rod 636 are unitary (i.e., formed of a single piece of material, such as stainless steel); but in other embodiments may be distinct pieces of material. In some embodiments spike distal end 644 can be configured as a conventional trocar tip, in which three or four planar surfaces converge at the spike distal end to form a point or chisel tip. However, particularly when the cartridge embodiment is primarily configured for separately forming a pilot hole with a distinct trocar, spike distal end 644 can alternatively be configured with conical point.

As with spike 408 described above, spike 632 has a length that is greater than a length (e.g., 66, 266) of a fastener with which tool cartridge 600 is configured to be used, such that the distal end extends beyond the fastener distal end to allow spike distal end 644 to guide the fastener into a pilot hole in bone tissue. In some embodiments, the spike length (from shoulder 648 to spike distal end 644) is between 3 mm and 8 mm longer than the length (e.g., 66, 266) of a fastener intended for use with the tool, such as, for example, between any two of: 3 mm, 4 mm, 5 mm, 6 mm, 7 mm, 8 mm, and/or 9 mm (e.g., between 4 mm and 6 mm) longer than the corresponding fastener length. For example, in the depicted embodiment, cartridge 600 is configured to deliver bone fasteners 210 with an overall length of 11.5 mm, and spike 632 is configured to extend 5 mm beyond fastener distal end, such that spike 408 has a length 452 of 16.5 mm (e.g., between 16 mm and 17 mm).

As described above for body 404, to facilitate insertion of a fastener and the ability of the shield to move relative to the body and fastener, the depicted configuration of cartridge 600 includes a tapered section 656 at shoulder 648. Specifically, tapered section 656 tapers from a nominal transverse dimension of the cartridge to the relatively smaller transverse dimension 644 at shoulder 648. In this configuration, dimension 644 is larger than the corresponding maximum transverse dimension of the fastener shaft (e.g., 130, 330) but smaller than the corresponding maximum transverse dimension of the outriggers (e.g., 134, 334), such that the outriggers are permitted a degree of flexibility in the longitudinal direction during insertion (e.g., to account for variations in underlying surfaces and facilitate full insertion of the fastener shaft and barbs). Of course, to facilitate the described movement of the shield assembly 416b (including shield 460b and hub 464b) relative to body 404b and a fastener disposed thereon, shield 460b has an inner transverse dimension (e.g., diameter) that is larger than corresponding maximum transverse dimensions of the fastener (e.g., dimension 134 or 334 of the outriggers) and of the body 404b. To facilitate user visibility during use, shield 460b is transparent in at least some embodiments to enable a user to perceive the position of the fastener and spike distal end relative to the distal end of the shield. For example, shield 460b may be formed of polycarbonate or other suitably durable polymers that are capable of being sterilized and/or are transparent or sufficiently translucent to permit a user to perceive the position of a fastener within the shield.

In use, distal end 424b of body 404b (without cartridge 600) is disposed at a position at which a user desires to insert a bone fastener (e.g., 210). A guidewire is then inserted through first handle passage 508 and first body passage 500 into bone tissue adjacent the targeted fastener position. The tool is then rotated around the guidewire, if needed, to align the second body passage 504 with the targeted fastener position, and a trocar 554 is inserted through second handle passage 512 and second body passage 508 pushed and/or rotated into the bone tissue to form a pilot hole in the bone tissue. In some embodiments, trocar 554 has a diameter (at its distal end) that is equal to or slightly smaller than the nominal diameter of the shaft (excluding barbs) of a fastener for which a pilot hole is drilled. For example, the trocar diameter may be between 85% and 100% (e.g., between 90% and 95%) of the fastener shaft diameter. FIG. 5D shows both the guidewire and trocar extending from body distal end 424b. Next, trocar 554 is removed, and body 404b retracted sufficiently to permit cartridge 600 to be laterally positioned over guidewire 550 with rod 636 aligned with second body passage 504. Cartridge 600 and tool body 404b are then moved longitudinally together such that rod 636 is inserted into first body passage 504 and cartridge projection 612 is inserted into recess 516 to engage tool body 404b. A user can then align spike 632 with the pilot hole in the underlying bone tissue, and advance the tool and cartridge toward the bone tissue, to drive the fastener (210) into the pilot hole and seat the fastener in the bone. If desired, shield assembly 416b can be moved to the extended position before positioning the fastener at or near the pilot hole (to protect the fastener and resist unintended engagement of the fastener with tissue during such positioning), and then shield assembly 416b can be retracted before or while the fastener is driven into the pilot hole.

With cartridges in a pre-loaded state (e.g., cartridge 600 with a fastener 210 pre-loaded on spike 632), the present kits can comprise a plurality of (e.g., three to five) cartridges 600 pre-loaded with bone fasteners (e.g., 210) and a single tool 400b. Some such kits can further comprise at least one guidewire 550 and/or at least one trocar 554. Such a kit can be sterile and enclosed in a sealed package (e.g., sealed tray or a blister pack).

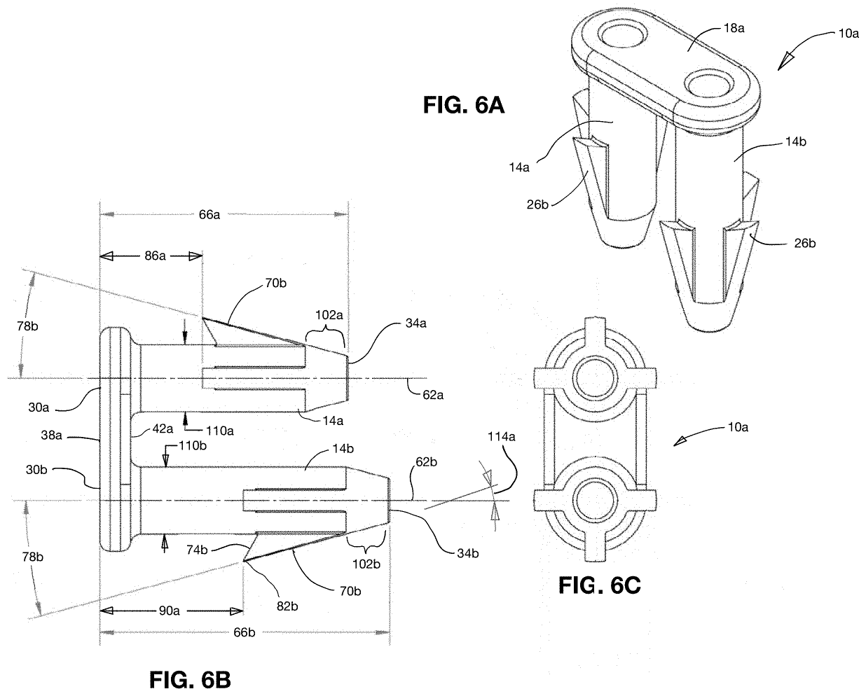

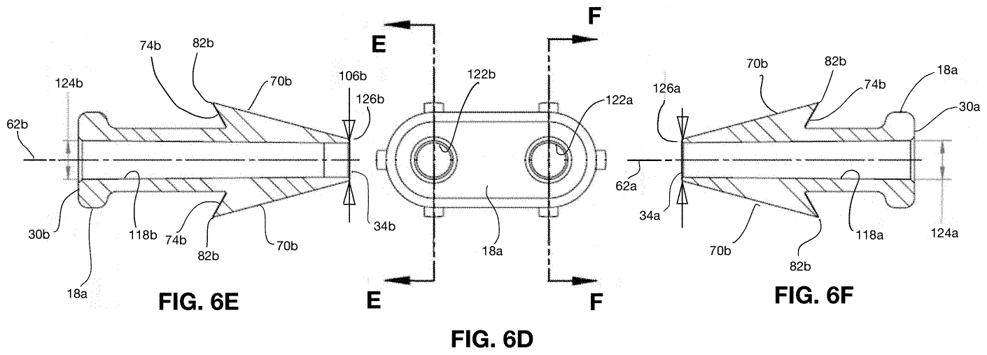

Referring now to FIGS. 6A-6G, a second embodiment 10a of the present soft-tissue fasteners is shown. In the depicted example, fastener 10a is configured for coupling an implant to soft tissue, such as a tendon (e.g., the supraspinatus tendon or rotator cuff). As shown, fastener 10a comprises two elongated shafts 14a, 14b, an enlarged head 18a, and a plurality of barbs 26b on each of the shafts 14a, 14b.

Each shaft 14a, 14b extends from a respective shaft proximal end 30a, 30b (and enlarged head 181) to a respective shaft distal end 34a, 34b. Shaft distal ends 34a, 34b are configured to be inserted (along with their respective barbs 26b) into soft tissue of a patient such that the barbs resist removal of the fastener from the soft tissue. For example, in at least some uses, the distal ends can first be inserted through an implant such that inserting the distal ends into soft tissue couples the implant to the soft tissue. To that end, enlarged head 18a extends between proximal ends 30a, 30b of the respective shafts to resist removal of such an implant over the proximal ends of the shafts. As shown, shaft 14b is longer than shaft 14a—i.e., length 66b of shaft 14b is larger than length 66a of shaft 14a. As a result, during insertion into soft tissue, distal end 34b of shaft 14b will enter the tissue before distal end 34a of shaft 14a, thereby reducing (relative to a similar structure with two similar shafts of the same length the cross-sectional area of fastener 10a being pushed into the tissue.

As shown, head 18a includes a head proximal side 38a, and a head distal side 42a. In this configuration, at least a portion of the head distal side is longitudinally aligned with shaft proximal ends 30a, 30b, for example as shown in FIG. 6B. In the embodiment shown, head 18a is substantially planar in that proximal side 38a is defined by a planar surface spanning a majority of a region between the shafts, and distal side 42a is defined by a planar surface spanning a majority of a region between the shafts.

As shown, each of barbs 26b extends longitudinally along a portion of a length 66a, 66b of the respective shaft. In this configuration, each barb extends linearly along the shaft, but in other embodiments, the barbs may be helical or otherwise curved or angled along the shaft. Each of the barbs has a respective distal leading edge 70b and a respective proximal trailing edge 74b that is spaced from the respective shaft proximal end 30a, 30b. For each barb, the leading edge faces distally (toward the respective shaft distal end 34a, 34b) and is disposed between the shaft distal end and the respective trailing edge. As shown, each of the leading edges is disposed at an acute angle relative to longitudinal axis 62a, 62b to facilitate insertion into tissue, and each of the trailing edges is also disposed at an acute angle relative to longitudinal axis 62a, 62b to resist removal of the fastener once inserted. In the embodiment shown, the leading edges of the barbs are disposed at a common angle relative to the respective longitudinal axis. For example, leading edges 70b of barbs 26b are disposed at an angle 78b. In some embodiments, angle 78b is between 10 and 20 degrees, for example, between any two of 10, 12.5, 15, 17.5, and/or 20 degrees (e.g., between 12.5 and 17.5 degrees). In the depicted embodiment, angle 78b is between 14.5 and 15.5 degrees. In other embodiments, the leading edge angles can be varied for different barbs, such as to manage the force required for insertion and minimize local tissue damage, while maintaining sufficient resistance to removal. For example, for a given barb length, as the angle of the leading edge increases, so does the distance the barb extends from the shaft and the resistance to removal. However, the farther the barb extends outward from the shaft, the greater the chances of tissue fibers being damaged by insertion of the fastener instead of simply permitting the barb to slip past those tissue fibers so they can be engaged by the barb's trailing edge. For example, in some embodiment, each shaft 14a, 14b can include a set of three barbs with three different leading edge angles, such as those used for fastener 10 described above.

Additionally, in the depicted embodiment, the trailing edges of the barbs on shaft 14b are staggered along the length of shaft relative to those on shaft 14a to increase the likelihood that at least some of the barbs will securely engage soft tissue. For example, when used with certain tendons (e.g., the supraspinatus tendon), the tendon may have different thicknesses in different regions or may have partial-thickness tears, such that barbs disposed at different distances from the head of the fastener make it more likely that at least one barb will encounter and engage soft tissue of sufficient integrity to resist removal of the fastener. More specifically, in the depicted embodiment, the longer second shaft 14b places trailing edges 74b on the first shaft closer to head 18a than are the barbs on first shaft 14a. As indicated in FIG. 6B, a proximal-most point or edge 82a of each barb 26b on shaft 14a is a distance 86a distal of shaft proximal end 30a, and a proximal-most point or edge 82a of each barb 26b on shaft 14b is a larger distance 90a distal of shaft proximal end 30b.

In the embodiment shown, barbs 26b on first shaft 14a are disposed at equiangular intervals around one half of that shaft, and barbs 26b on second shaft 14b are disposed at equiangular intervals around an opposing half of that shaft (i.e., such that two of the barbs on a shaft are separated by 180 degrees, and the barbs on each shaft do not extend toward the other shaft). Specifically, on shaft 14a, three barbs 26b are disposed at angular intervals of 90 degrees and on shaft 14b, three barbs 26b are disposed at angular intervals of 90 degrees. In other embodiments with a different number of barbs and equiangular intervals, the equiangular intervals will necessarily also vary (e.g., four barbs in one half of a shaft perimeter would be disposed at angular intervals of 60 degrees. In other embodiments, barbs may be disposed at different angular intervals around the shaft.

To further facilitate insertion through an implant and into soft tissue, an outer surface 98a, 98b of each shaft defines a tapered section 102a, 102b adjacent a respective one of shaft distal ends 34a, 34b. Each tapered section 102a, 102b extends from a first outer transverse dimension 106a, 106b at the respective shaft distal end (34a, 34b) and increases in size in a proximal direction to a larger outer transverse dimension 110a, 110b that may, as in the depicted embodiment, be substantially equal to an outer transverse dimension along a remainder of the length of the shaft between the tapered section and the head (18a). In the embodiment shown, each tapered section 102a, 102b is tapered linearly at a taper angle 114a relative to respective axis 62a, 62b, but in other embodiments may be tapered in a non-linear fashion (e.g., along a curved path). Angle 114b may in some embodiments be equal to angle 78b, and/or may be between 10 and 20 degrees, for example, between any two of 10, 12.5, 15, 17.5, and/or 20 degrees (e.g., between 12.5 and 17.5 degrees). For example, in the depicted embodiment, angle 114a is between 14.5 and 15.5 degrees, and is equal to angle 78b such that tapered section 102 and leading edge 70b follow a continuous, linear path.

In the embodiment shown, each shaft (14a, 14b) is also configured to receive a portion of a delivery tool (described below). In particular, an internal surface 118a, 118b of the respective shaft defines an internal passage 122a, 122b that extends through the length of the shaft along axis 62a, 62b and through both of the respective proximal and distal ends. Each passage 122a, 122b is configured to receive a portion of a tool (e.g., a spike or trocar) such that that the tool extends through passages 122a, 122b and beyond distal ends 34a, 34b to permit the tool to form a pilot hole in the tissue into which the tool (or a user of the tool) can simultaneously drive the fastener and, after the fastener is inserted into the tissue, the tool can thereafter be removed via the passages (122a, 122b). In some embodiments, each passage 122a, 122b has a first inner transverse dimension at the respective shaft proximal end (30a, 30b) and a smaller second inner transverse dimension at the respective shaft distal end (34a, 34b), such that the passage tapers (e.g., linearly) from smaller at the shaft distal end to larger as the passage approaches the shaft proximal end. For example, in the embodiment shown, each passage 122a, 122b tapers linearly at an angle 124a, 124b, each of one degree. Such a taper can be advantageous in reducing the resistance to removal of a tool after insertion of the fastener. For example, during insertion of the fastener, soft tissue will be displaced and tend to exert an inward force around the perimeter of a tool in the passage. The provision of a taper, such that a proximal portion of the passage is larger than a distal portion of the passage, typically helps mitigate the compressive forces on a tool in the passage during insertion of the fastener and thereby typically reduces resistance to removal of the tool from the passage after such insertion. In some embodiments, such as the one shown, each inner surface 118a, 118b and respective outer surface 98a, 98b meet at the respective shaft distal end 34a, 34b to form an edge 126a, 126b. Such an edge at the distal end of the shaft minimizes the cross-sectional area of fastener at the distal end to facilitate insertion of the fastener into tissue.

In the embodiment of FIGS. 6A-6F, each shaft 14a, 14b has a circular cross-sectional shape, such that an outer transverse dimension 110a, 110b is the diameter of a circle that defines the outer perimeter of that shape. In some embodiments, transverse dimensions 110a, 110b are each between any two of: 1.25 mm, 1.35 mm, 1.45 mm, 1.55 mm, 1.65 mm, 1.75 mm, 1.85 mm, 1.95 mm, and/or 2.05 mm (e.g., between 1.55 mm and 1.75 mm). For example, in the embodiment shown, diameters 110a, 110b are each between 1.6 and 1.7 mm. In other embodiments, the shaft can have other cross-sectional shapes, such as, for example, square, hexagonal, octagonal, or the like.

As described above, the barbs of the fastener extend outwardly from the shaft and therefore span a maximum transverse dimension that is larger than that of the shaft, such as, for example, a maximum transverse dimension that is between any two of: 130%, 140%, 150%, 160%, 170%, 180%, 190%, 200%, 210%, 220%, and/or 230% of the maximum transverse dimension of the shaft (e.g., between 170% and 190% of the diameter of the corresponding shaft). For example, in the embodiment shown, barbs 26b each extend radially outward to a point between 1.4 and 1.6 mm from the respective axis 62a, 62b (i.e., each of point/edge 82b is between 1.4 and 1.6 mm from the respective axis 62a, 62b).

In some embodiments, the overall length of the fastener is between 5 mm and 9 mm, such as, for example, between any two of 5 mm, 5.5 mm, 6 mm, 6.5 mm, 7 mm, 7.5 mm, 8 mm, and/or 8.5 mm (e.g., between 5.5 mm and 6.5 mm, between 6.5 mm and 7.5 mm, or between 7.5 mm and 8.5 mm). For example, in the embodiment shown, length 66b second shaft 14b is nominally 7.1 mm, and therefore between 6.5 mm and 7.5 mm, and length 66a of first shaft 14a is nominally 6.1 mm. In other embodiments, lengths 66a, 66b can be selected for a particular use. For example, when using a soft-tissue fastener with a tendon that overlies and must move relative to other anatomical structure, it is generally desirable that the distal end of the fastener not extend into the underlying anatomical structures. As an example of one type of tendon, for use with a relatively smaller person with a relatively thinner supraspinatus tendon, a fastener with maximum nominal length 66b of 6 mm may be appropriate; while, for use with a relatively larger person with a relatively thicker supraspinatus tendon, a fastener with a maximum nominal length 66b of 8 mm may be appropriate.

In at least some embodiments, the fastener (i.e., shafts 14a and 14b, head 18a, and barbs 26a) are defined by a unitary piece of material. For example, it is sometimes desirable for soft-tissue fasteners to be bioresorbable and, in such instances, the fastener can be molded of a bioresorbable polymer such as poly lactic-co-glycolic acid (PLGA) or polylactic acid (PLA or PLLA). In some uses, it is desirable for complete resorption to occur in less than 9 months (e.g., between 3 months and 9 months).

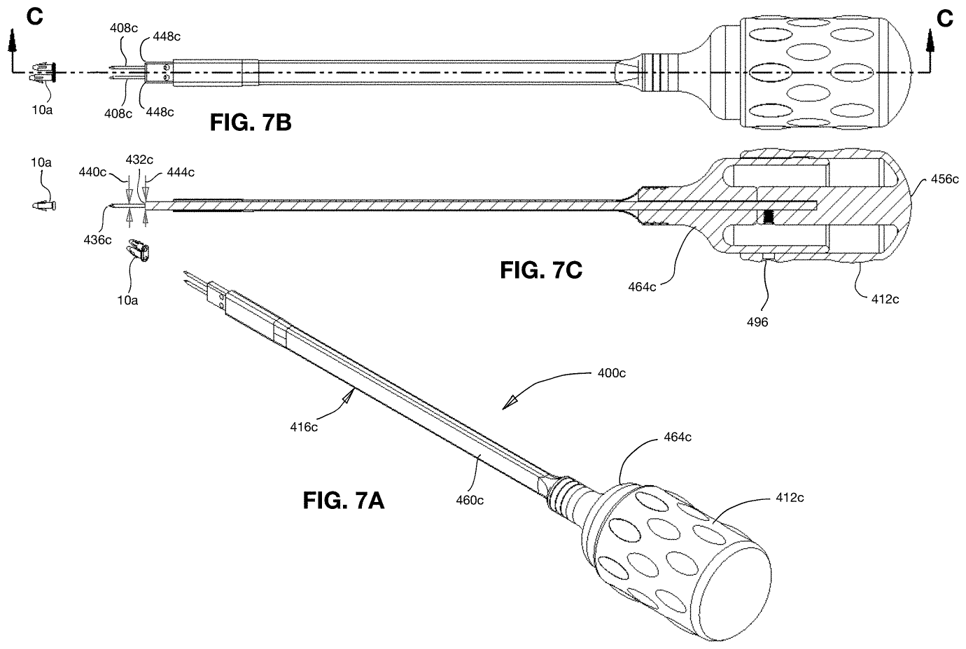

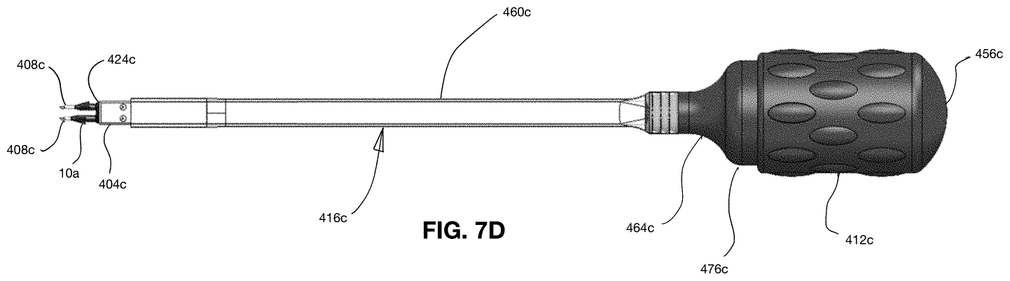

Referring now to FIGS. 7A-7D, an embodiment of a tool 400c for delivery of a fastener 10a of FIG. 6A is shown, with a fastener 10a shown adjacent a distal end of the tool (FIGS. 7A-7C) or coupled to a distal end of the tool (FIG. 7D). Tool 400c is substantially similar to tool 400a, with the primary difference being that tool 400c comprises two spikes 408c that are spaced apart and parallel to each other to receive a fastener 10a with one of spikes 408c in each of passages 122a, 122b of respective fastener shafts 14a, 14b. Of course, to facilitate use of tool 400c to drive a fastener into soft tissue, each spike proximal end 432c has a transverse dimension 440c that is smaller than a corresponding transverse dimension 444c of the body distal end such that a shoulder 448c is defined at each spike proximal end 432c. And, as described above for shoulder 448 of tool 400a, the shoulder(s) (448c) is/are configured to abut a head of a fastener received over the pair of spikes.

As described above for tool 400a, tool 400c is also particularly well-suited for implementations in which the tool is provided to a user with the tool pre-loaded with a corresponding fastener (e.g., 10a). For example, the longitudinal overlap of hub 464a and handle 412a facilitates the inclusion of a frangible pin 496 (shown broken in FIG. 7C) that extends through the handle and into or proximal to the hub when the shield is in the extended position (in which distal end 472c of shield 460c extends beyond distal ends 436c of spikes 408c) to resist movement of the shield until a user applies sufficient longitudinal compressive force between the handle and the hub to break the pin, and thereby retract the shield and expose the fastener. In other embodiments, instead of the frangible pin, a detent or other structure can be used that resists inadvertent retraction of the shield but still permits a user to overcome that resistance by hand when ready to deploy the fastener. With tools 400c in a pre-loaded state, the present kits can comprise a plurality of (e.g., three to five) soft-tissue tools (e.g., 400c) pre-loaded with soft-tissue fasteners (e.g., 10a) and having their respective shield assemblies in an extended position, and/or a plurality of (e.g., six to eight) bone tools (e.g., 400a) pre-loaded with bone fasteners (e.g., 210a, described below) and having their respective shield assemblies in an extended position. Such a kit can be sterile and enclosed in a sealed package (e.g., sealed tray or a blister pack).

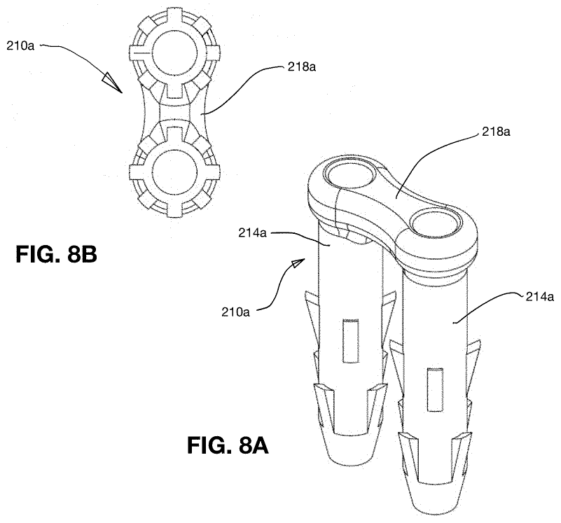

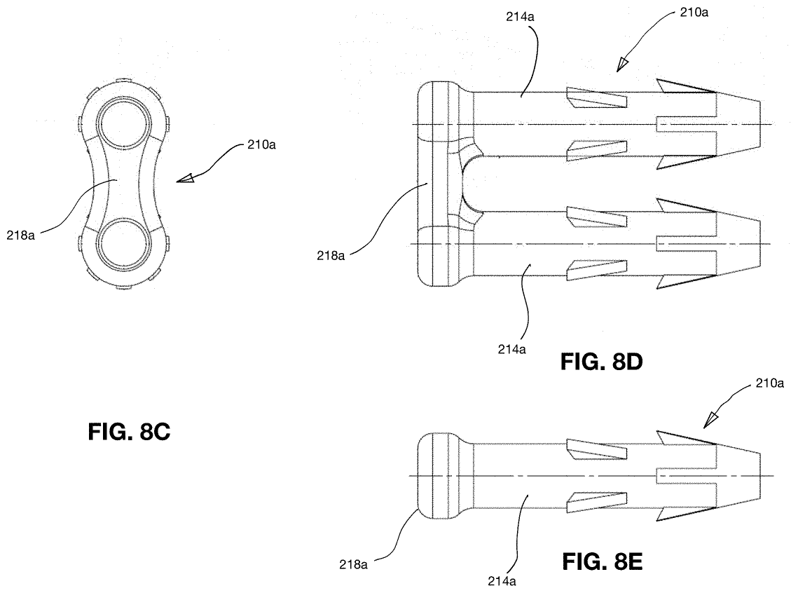

Referring now to FIGS. 8A-8C, a second embodiment 210a of the present bone fasteners is shown. Fastener 210a is substantially similar to fastener 210 with the primary difference being that fastener 210a includes two shafts 214a and an enlarged head 218a that spans both shafts 214a, and that fastener 210a omits outriggers 22. Each of shafts 214a is substantially similar to shaft 214.

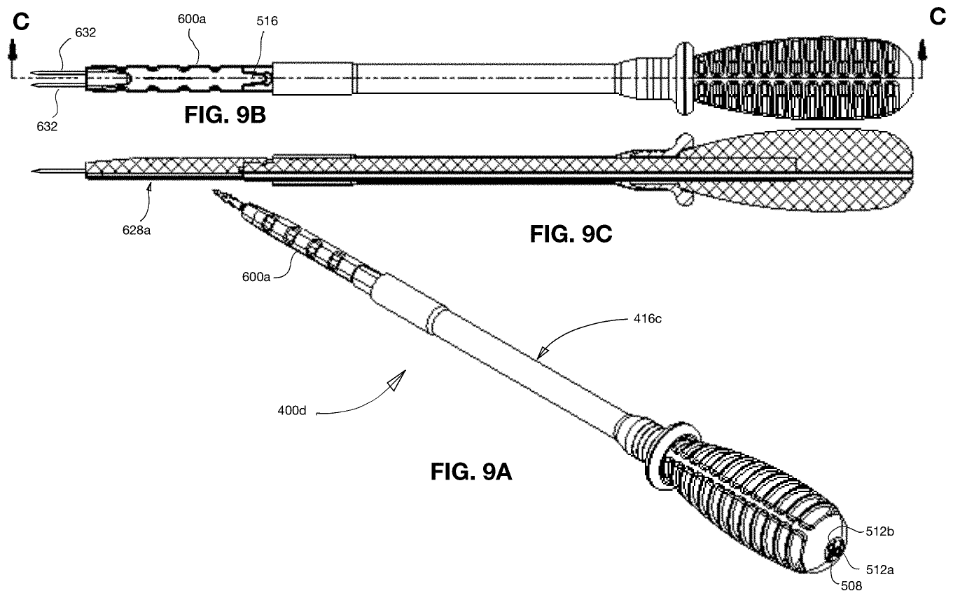

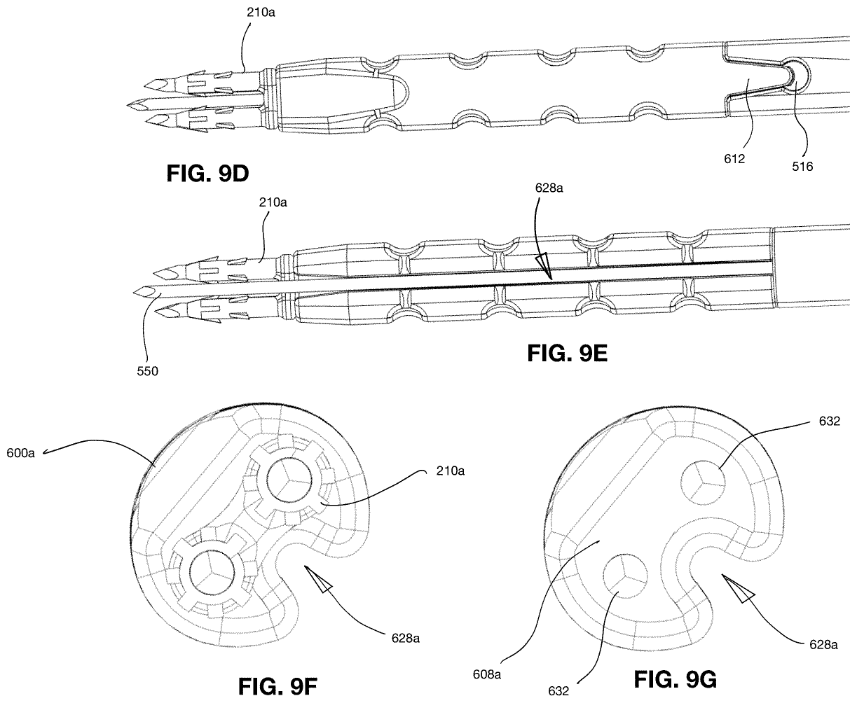

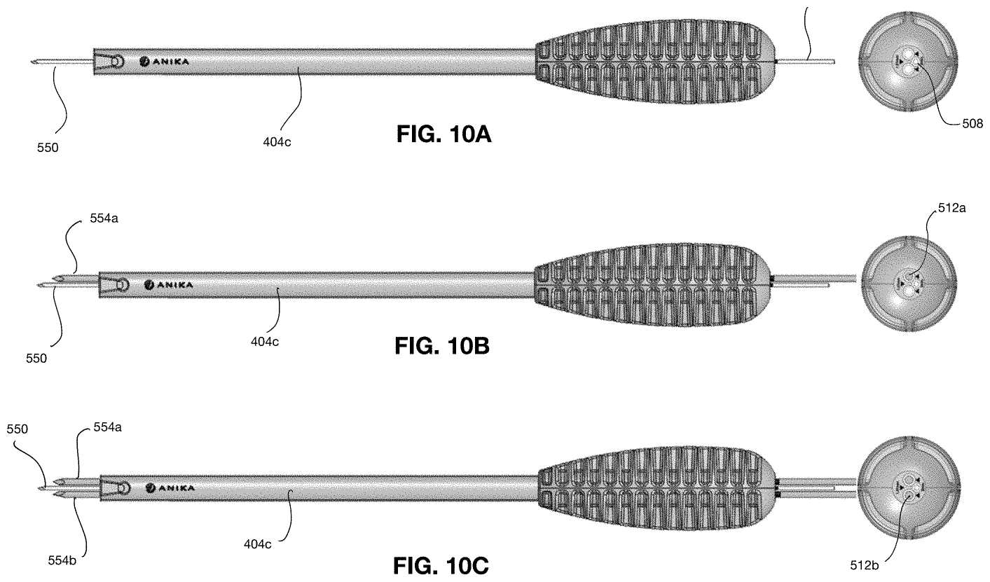

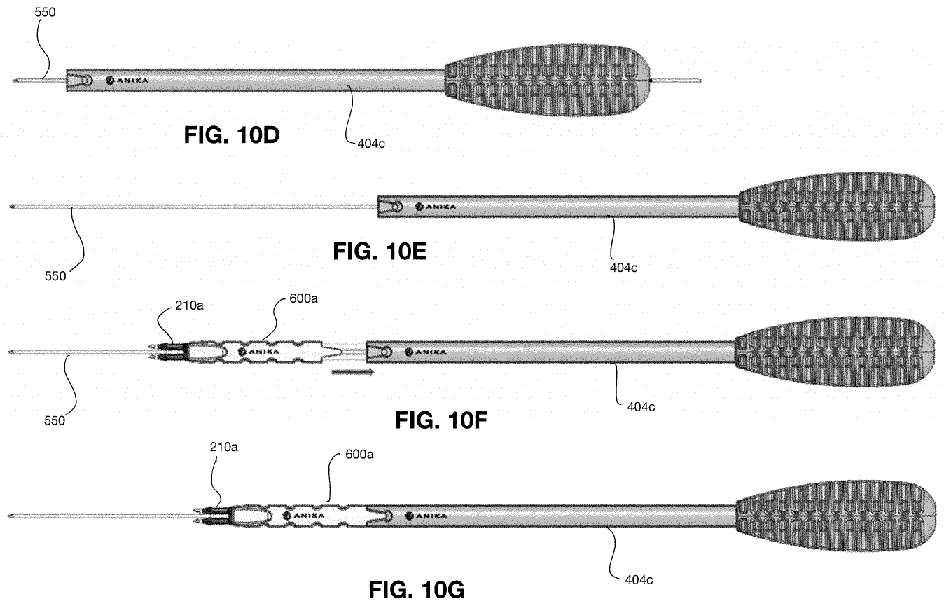

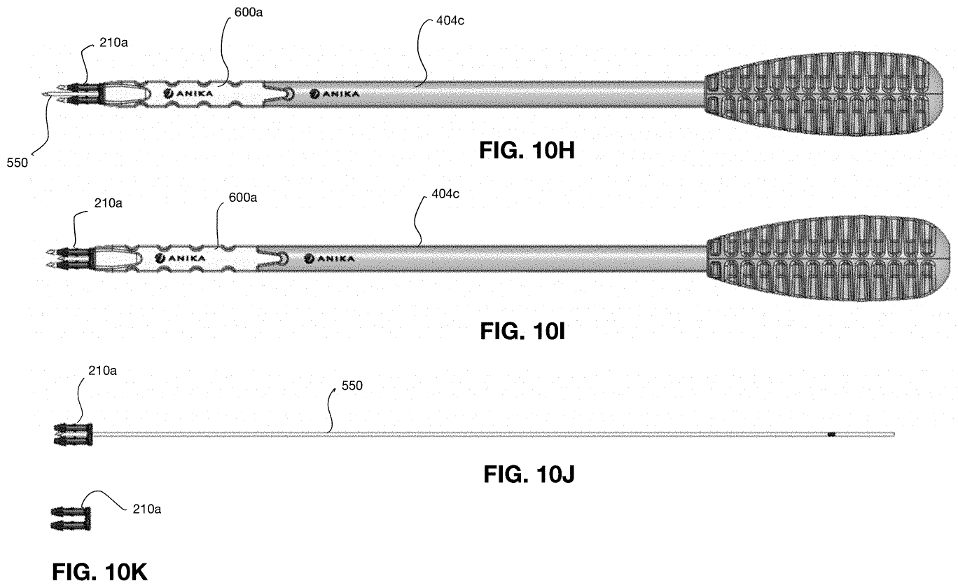

Referring now to FIGS. 9A-9G and FIGS. 10A-10K; FIGS. 9A-9G depict an embodiment of a tool 400d for delivery of a fastener 210a via a cartridge 600a, with a fastener 210a coupled to a distal end of the cartridge (FIGS. 9A, 9D-9F), and the cartridge coupled to a distal end of the tool; and FIGS. 10A-10K depict an exemplary sequence of steps for deployment of a fastener 210a via tool 400d.

Tool 400d is substantially similar to tool 400b, with the primary exception being that tool 400d includes two trocar passages aligned with handle passages 512a, 512b in addition to guidewire aligned with handle passage 508.

Cartridge 600a is substantially similar to cartridge 600, with the primary exception being that cartridge 600a includes two spikes 632, and two rods 636, and that groove 628a opens laterally in a direction that opposes projection 612 and is perpendicular to a plane passing through both of spikes 632a, 632b. As shown, spikes 632 are spaced apart and parallel to each other to receive a fastener 210a with one of spikes 632 in each of passages 322 of respective fastener shafts 214a, 214b. As described above for shoulder 648 of cartridge 600, shoulder(s) (648a) is/are configured to abut a head of a fastener received over the pair of spikes.

In use, distal end 424c of body 404c (without cartridge 600a) is disposed at a position at which a user desires to insert a bone fastener (e.g., 210a). A guidewire 550 is then inserted through first handle passage 508 and the corresponding guidewire passage in body 404c into bone tissue adjacent the targeted fastener position (FIG. 10A). The tool is then rotated around the guidewire, if needed, to align the trocar passages (and handle passages 512a, 512b) with the targeted fastener position, and a first trocar 554a is inserted through handle passage 512a and the corresponding passage in body 404c, and pushed and/or rotated into the bone tissue to form a first pilot hole in the bone tissue (FIG. 10B). A second trocar 554b is inserted through handle passage 512b and the corresponding passage in 404c, and pushed and/or rotated into the bone tissue to form a second pilot hole in the bone tissue (FIG. 10C). Next, trocars 554a, 554b are removed (FIG. 10D), and body 404c retracted in a proximal direction sufficiently to permit cartridge 600a to be laterally positioned over the guidewire with guidewire 550 in groove 628a with rods 636 aligned with the corresponding passages in body 404c (FIG. 10F). Cartridge 600a and tool body 404b are then moved longitudinally together such that rods 636 are inserted into the corresponding passages in body 404c and cartridge projection 612 is inserted into recess 516 to engage tool body 404c (FIG. 10G). A user can then align spikes 632 with the pilot holes in the underlying bone tissue, and advance the tool and cartridge toward the bone tissue (FIG. 10H), to drive the fastener (210) into the pilot hole and seat the fastener in the bone (FIG. 10I). If desired, shield assembly 416c (FIGS. 9A-9C) can be moved to the extended position before positioning the fastener at or near the pilot hole (to protect the fastener and resist unintended engagement of the fastener with tissue during such positioning), and then shield assembly 416c can be retracted before or while the fastener is driven into the pilot hole. The tool can then be retracted in a proximal direction to slide the tool 404c and cartridge 600a over and off of guidewire 550 (FIG. 10J), after which the guidewire can be removed, leaving fastener 210 in place (FIG. 10K).

With cartridges in a pre-loaded state (e.g., cartridge 600a with a fastener 210a pre-loaded on spikes 632), the present kits can comprise a plurality of (e.g., three to five) cartridges 600a pre-loaded with bone fasteners (e.g., 210a) and a single tool 400c. Some such kits can further comprise at least one guidewire 550 and/or at least two trocars 554. Such a kit can be sterile and enclosed in a sealed package (e.g., sealed tray or a blister pack).

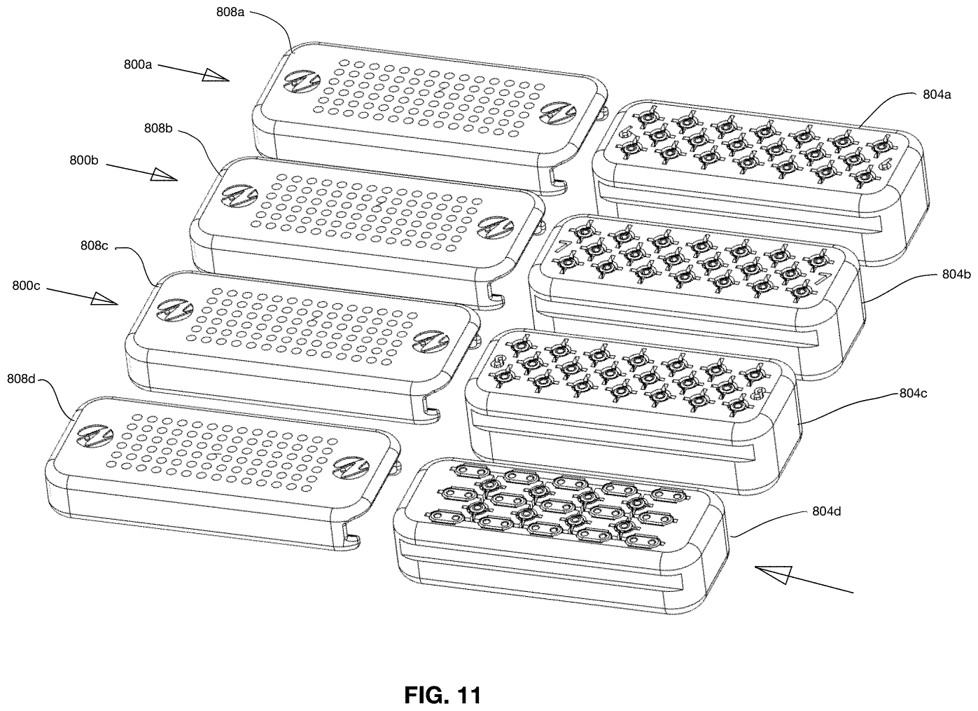

Referring now to FIG. 11, several examples of trays or caddies 800a, 800b, 800c, 800d are shown, each configured to hold a plurality of the present fasteners (e.g., 10, 10a, 210, 210a). Each of the trays or caddies includes a respective base 804a, 804b, 804c, 804d configured to receive the respective plurality of fasteners, and a respective lid or cover 808a, 808b, 808c, 808d to enclose the fasteners. Each of trays/caddies 800a, 800b, 800c are configured to hold a plurality of single-shaft fasteners (e.g., 10 and/or 210), while tray/caddy 800d is configured to hold a plurality of single-shaft fasteners (e.g., 10 and/or 210) and a plurality of dual-shaft fasteners (e.g., 10a and/or 210a). As shown, the bases are configured to receive and support the fasteners with the respective fastener proximal ends (e.g., 30, 30a, 230, 230a) facing upward to facilitate loading each fastener onto a spike of a corresponding tool.

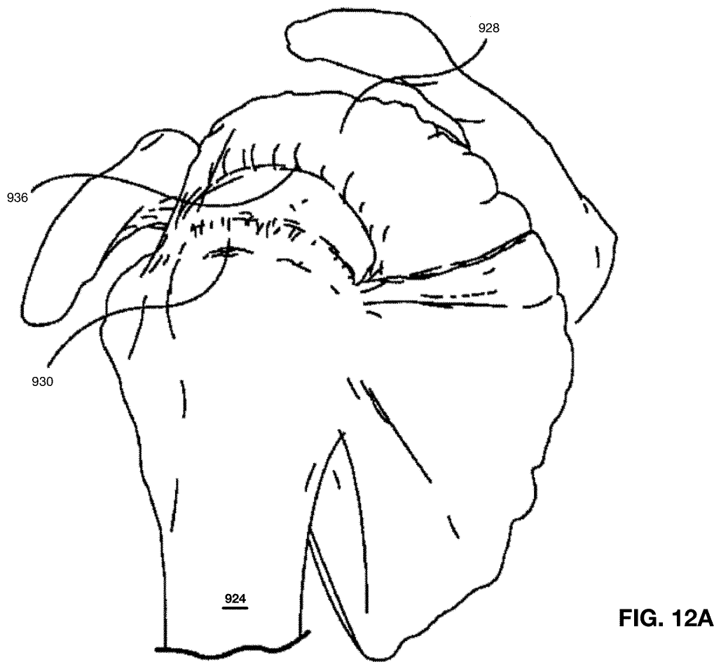

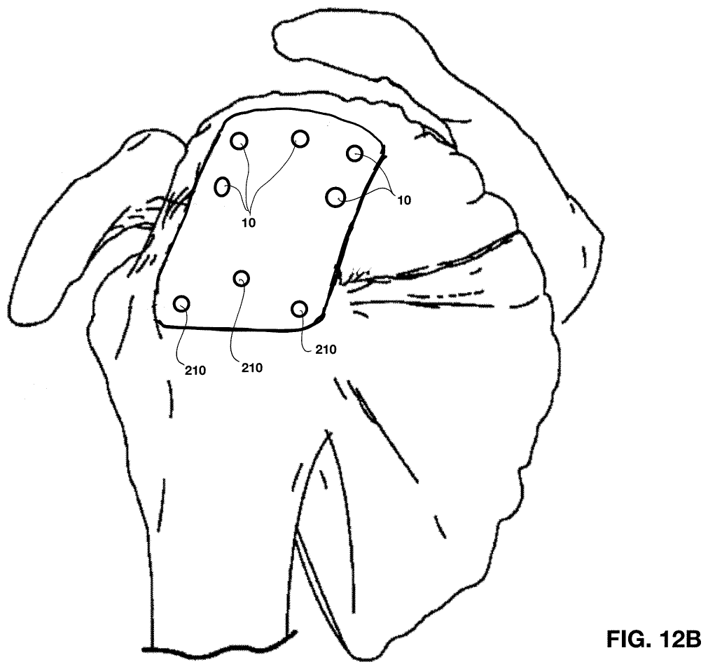

Referring now to FIGS. 12A and 12B, FIG. 12A depicts a perspective, cutaway view of the connection of a damaged supraspinatus tendon to the humerus in a human shoulder; and FIG. 12B depicts a perspective, cutaway view of the connection of a damaged supraspinatus tendon to the humerus in a human shoulder, showing a repair in accordance with an example of the present methods.

As shown in FIG. 12A, and described in United States Patent Application No. US 2008/0188936, the rotator cuff is the complex of four muscles that arise from the scapula and whose tendons blend in with the subjacent capsule as they attach to the tuberosities of the humerus. The subscapularis arises from the anterior aspect of the scapula and attaches over much of the lesser tuberosity. The supraspinatus muscle arises from the supraspinatus fossa of the posterior scapula, passes beneath the acromion and the acromioclavicular joint, and attaches to the superior aspect of the greater tuberosity. The infraspinatus muscle arises from the infraspinous fossa of the posterior scapula and attaches to the posterolateral aspect of the greater tuberosity. The teres minor arises from the lower lateral aspect of the scapula and attaches to the lower aspect of the greater tuberosity. Proper functioning of the rotator, 3 to 4 millimeters thick, depends on the fundamental centering and stabilizing role of the humeral head with respect to sliding action during anterior and lateral lifting and rotation movements of the arm.

In rotator cuff injuries, and shown in FIG. 12A, suprasinatus 928 frequently tears away from the humerus 924 due to high stress activity or traumatic injury. Supraspinatus 928 has separated from humerus 924 along its lateral edge 936 away from its attachment surface or “footprint” in the greater tuberosity 930.

As shown in FIG. 12B, the present methods of repairing a torn rotator cuff, and in some particular implementations, a torn supraspinatus tendon can comprise disposing an implant (e.g., a fibrous implant) 940 over a torn supraspinatus tendon 928; securing a first portion of the implant 940 to the supraspinatus tendon 928 with a plurality of soft-tissue fasteners (e.g., 10, 10a), for example using one of the present tools (e.g., 400, 400a, 400b, 400c); and securing a second portion of implant 940 to the humerus (924) with a plurality of bone fasteners (e.g., 210, 210a), for example using one of the present tools (e.g., 400, 400a, 400b, 400c). In some implementations of the present methods, the first portion of the implant 940 (e.g., an edge of the first portion of implant 940) is also sutured to the supraspinatus tendon, for example, to ensure more-continuous engagement between the implant and the tendon and thereby encourage tissue growth to and into the implant.

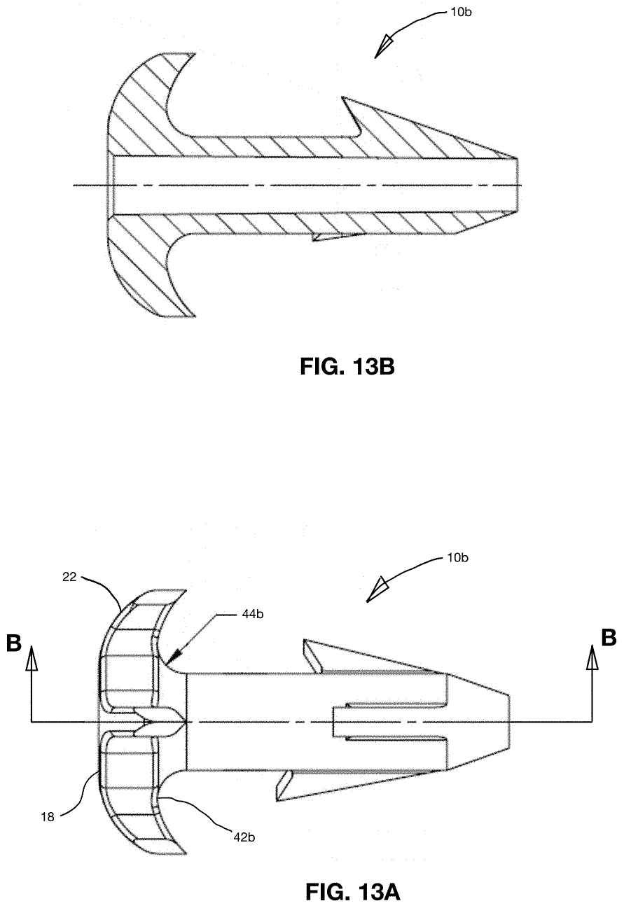

Referring now to FIGS. 13A and 13B, FIG. 13A depicts a side view of a third embodiment 10b of the present soft-tissue fasteners, and FIG. 13B depicts a side cross-sectional view of fastener 10b, taken along the plane B-B of FIG. 13A. Fastener 10b is substantially similar to fastener 10 of FIGS. 1A-1H, with the primary difference being that distal side 42b of head 18b is entirely concave and forms a continuous concave surface with the distal side of outriggers 22, as shown. Specifically, in the depicted embodiment, a radius 44b forms a concave surface that meets the nominal diameter of shaft 14.

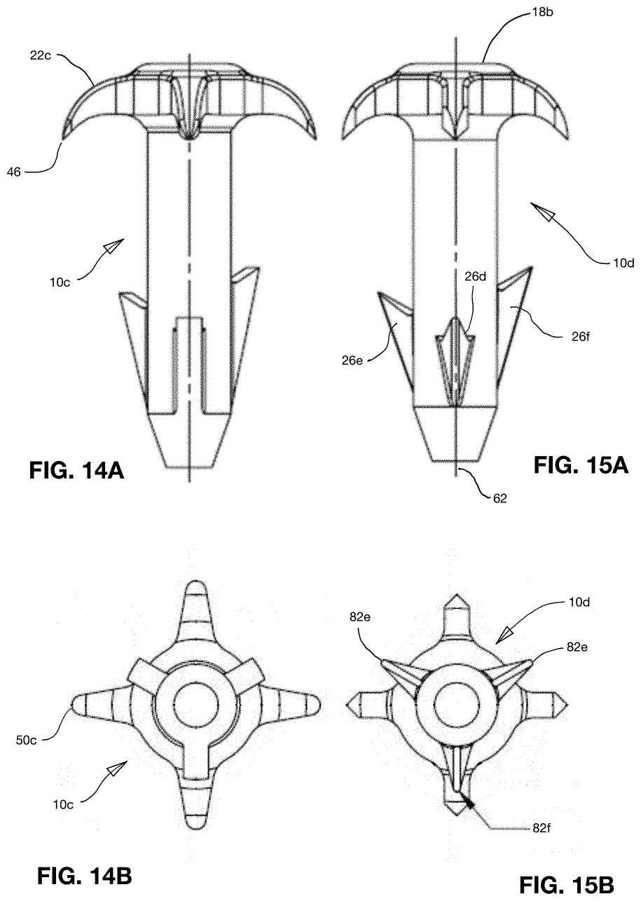

Referring now to FIGS. 14A and 14B, FIG. 14A depicts a side view of a fourth embodiment 10c of the present soft-tissue fasteners, and FIG. 14B depicts a distal end view of fastener 10c. Fastener 10c is substantially similar to fastener 10b of FIGS. 13A-13B, with the primary difference being that each outrigger 22c of fastener 10c taper to a distal end 46 that is not pointed (omits point 54) and instead curves to an edge 50c that is oriented substantially radially rather than longitudinally. In this configuration of outriggers 22c, distal ends 46c are less likely to tear or cut into an implant or underlying tissue.

Referring now to FIGS. 15A and 15B, FIG. 15A depicts a side view of a fifth embodiment 10d of the present soft-tissue fasteners, and FIG. 15B depicts a distal end view of fastener 10d. Fastener 10d is substantially similar to fastener 10b of FIGS. 13A-13B, with the primary differences being that barbs 26d, 26e, 26f are shaped differently than barbs 26a, 26b, 26c, and that barbs 26d, 26e, 26f extend farther radially outward from longitudinal axis 62 than do barbs 26a, 26b, 26c. More particularly, barbs 26d, 26e, 26f each have a substantially triangular profile. Specifically, unlike barbs 26a, 26b, 26c that are each defined by a respective pair of planar sides that are parallel to each other (see FIG. 1B), barbs 26d, 26e, 26f are each defined by a respective pair of planar sides that angle toward each other (both laterally and longitudinally, such that each barb narrows with increasing distance from axis 62 and with increasing distance from head 18) and are joined by a radius that provides a curved outer surface along the apex of the respective barb (see FIG. 15B) rather than an edge. This substantially triangular cross-sectional profile is configured to facilitate insertion of the fastener by reducing insertion force (relative to a rectangular profile with a similar radial dimension), and the absence of a sharp edge configured to reduce the likelihood that the barbs will cut a fabric like implant through which the fastener is inserted.

As described above for fastener 10, the barbs of fastener 10d extend outwardly from the shaft and therefore extend to an imaginary circle (perpendicular to axis 62) having a maximum transverse dimension that is larger than that of the shaft. For fastener 10d, however, that maximum transverse dimension is between any two of: 160%, 170%, 180%, 190%, 200%, 210%, 220%, 230%, 240%, 250%, 260%, and/or 270% of the maximum transverse dimension of the shaft (e.g., between 210% and 220% of the diameter of the shaft). For example, in the embodiment shown, barbs 26d, 26e, 26f each extend radially outward to a point between 1.7 and 1.8 mm from axis 62 (i.e., each of point/edge 82d, 82e, 82f is between 1.7 and 1.8 mm from axis 62). This increased radial dimension is configured to engage more tissue and resist removal of the fastener from tissue. While the increased radial dimension also increases the force required for insertion (relative to a similar profile with smaller radial dimension), the substantially triangular profile discussed above offsets the increase due to the larger radial extent and ultimately makes for an insertion force similar to or slightly less than that of fastener 10.

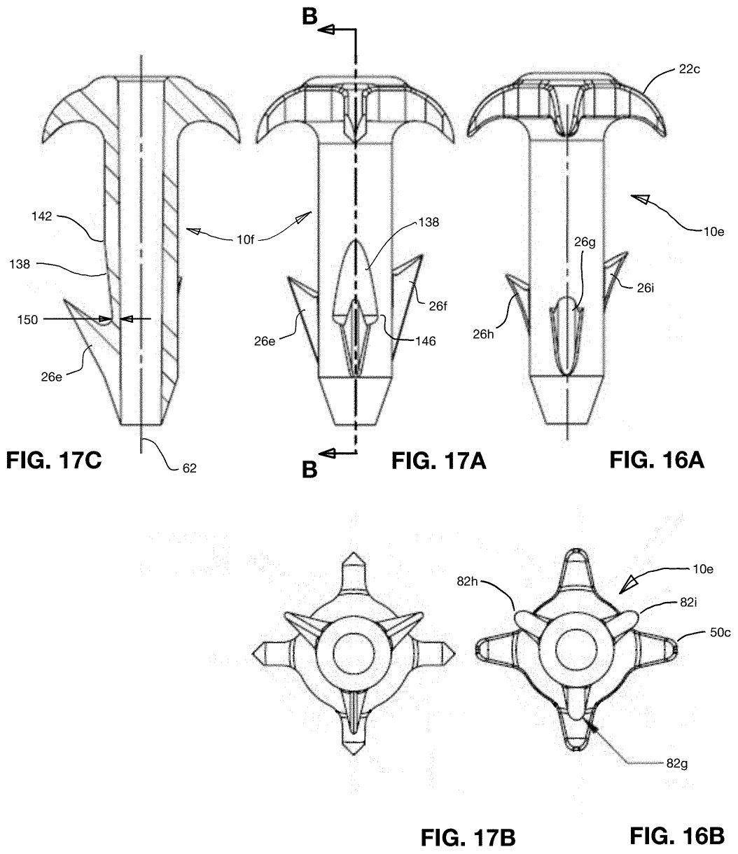

Referring now to FIGS. 16A and 16B, FIG. 16A depicts a side view of a sixth embodiment 10e of the present soft-tissue fasteners, and FIG. 16B depicts a distal end view of fastener 10e. Fastener 10e is substantially similar to fastener 10b of FIGS. 13A-13B, with the primary differences being that barbs 26g, 26h, 26i are shaped differently than barbs 26a, 26b, 26c, that barbs 26d, 26e, 26f extend farther radially outward from longitudinal axis 62 than do barbs 26a, 26b, 26c, and that the fastener includes outriggers 22c like those of fastener 10c of FIGS. 14A-14B. More particularly, barbs 26g, 26h, 26i each have a substantially triangular profile like that of barbs 26d, 26e, 26f, except barbs 26g, 26h, 26i have a larger radius at their respective apexes. As with barbs 26d, 26e, 26f, the cross-sectional profile of barbs 26g, 26h, 26i is configured to facilitate insertion of the fastener by reducing insertion force relative to a rectangular profile with a similar radial dimension, and the absence of a sharp edge configured to reduce the likelihood that the barbs will cut a fabric like implant through which the fastener is inserted.

As described above for fastener 10, the barbs of fastener 10e extend outwardly from the shaft and therefore extend to an imaginary circle (perpendicular to axis 62) having a maximum transverse dimension that is larger than that of the shaft. For fastener 10e, however, that maximum transverse dimension is between any two of: 140%, 150%, 160%, 170%, 180%, 190%, 200%, 210%, 220%, 230%, and/or 240% of the maximum transverse dimension of the shaft (e.g., between 180% and 190% of the diameter of the shaft). For example, in the embodiment shown, barbs 26dg, 26h, 26i each extend radially outward to a point between 1.5 and 1.6 mm from axis 62 (i.e., each of point/edge 82g, 82h, 82i is between 1.5 and 1.6 mm from axis 62). This increased radial dimension is configured to engage more tissue and resist removal of the fastener from tissue. While the increased radial dimension also increases the force required for insertion (relative to a similar profile with smaller radial dimension), the profile discussed above offsets the increase due to the larger radial extent and ultimately makes for an insertion force similar to or slightly less than that of fastener 10.

Referring now to FIGS. 17A-17B, FIG. 17A depicts a side view of a seventh embodiment 10f of the present soft-tissue fasteners, FIG. 17B depicts a distal end view of fastener 10f, and FIG. 19C depicts a side cross-sectional view of fastener 10f, taken along the plane C-C of FIG. 17A. Fastener 10f is substantially similar to fastener 10d of FIGS. 15A-15B, with the primary difference being that fastener 10f includes hollowed portions 138 extending into shaft 14f at the base of each barb 26d, 26e, 26f. More particularly, each hollowed portion 138 is defined by a planar surface extending from a proximal end 142 that is a first distance (equal to diameter 130) from axis 62, and a distal end 146 that is a second distance 150 from axis 62, with second distance 146 being less than diameter 130 such that the planar surface tapers toward axis 62 with increasing distance from head 18b. In this configuration, the radial distance of the proximal side of each barb available to engage tissue is increased without increasing the maximum radial extent of the barb. In other embodiments surface 138 may be non-planar (e.g., curved). In the embodiment shown, distal end 146 of each hollowed portion is joined to the proximal side of the respective barb via a curve or radius.

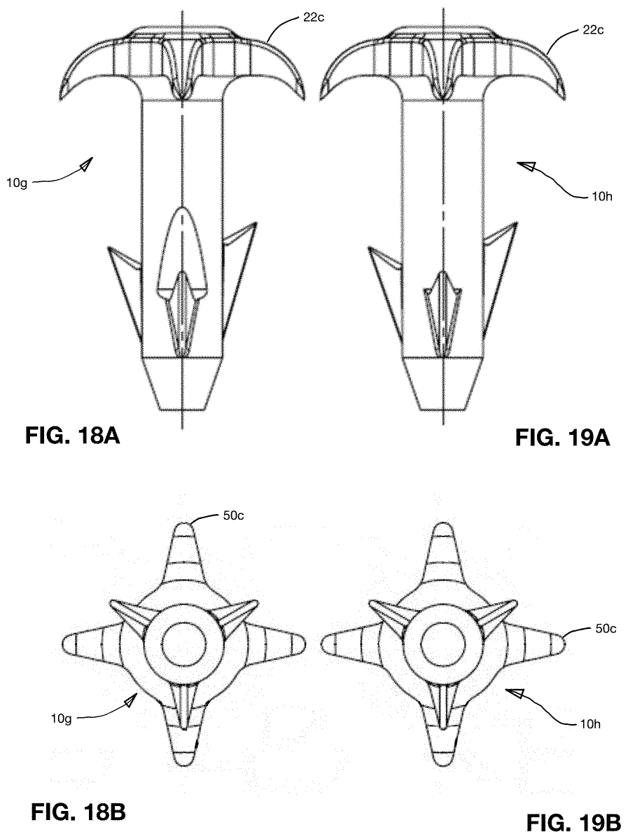

Referring now to FIGS. 18A and 18B, FIG. 18A depicts a side view of an eighth embodiment 10g of the present soft-tissue fasteners, and FIG. 18B depicts a distal end view of fastener 10g. Fastener 10g is substantially similar to fastener 10f of FIGS. 17A-17C, with the primary differences being that fastener 10g includes outriggers 22c that like those of fastener 10c of FIGS. 14A-14B.

Referring now to FIGS. 19A and 19B, FIG. 19A depicts a side view of a ninth embodiment 10h of the present soft-tissue fasteners, and FIG. 19B depicts a distal end view of fastener 10h. Fastener 10h is substantially similar to fastener 10d of FIGS. 15A-15B, with the primary differences being that fastener 10h includes outriggers 22c that like those of fastener 10c of FIGS. 14A-14B.

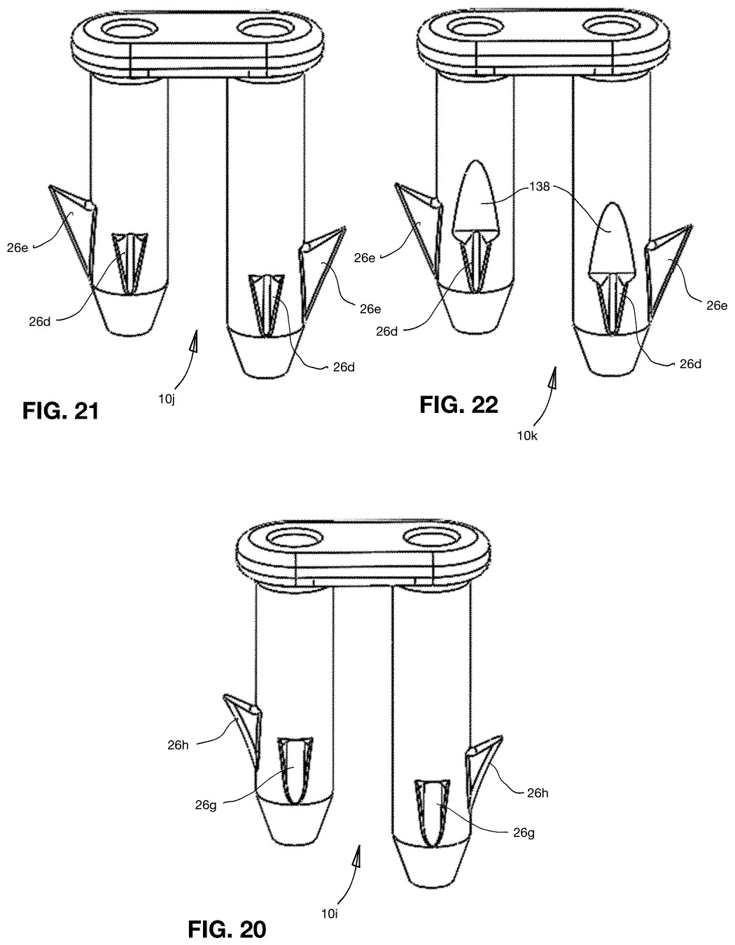

Referring now to FIG. 20, shown therein is a tenth embodiment 10i of the present soft-tissue fasteners. Fastener 10i is substantially similar to 10a of FIGS. 6A-6F, with the primary difference being that each of shafts 14a, 14b includes barbs 26g, 26h, 26i (not shown but 180° opposite barb 26g) like those of fastener 10e of FIGS. 16A-16B, instead of three barbs 26b.

Referring now to FIG. 21, shown therein is an eleventh embodiment 10j of the present soft-tissue fasteners. Fastener 10j is substantially similar to 10a of FIGS. 6A-6F, with the primary difference being that each of shafts 14a, 14b includes barbs 26d, 26e, 26f (not shown but 180° opposite barb 26d) like those of fastener 10d of FIGS. 15A-15B), instead of three barbs 26b.

Referring now to FIG. 22, shown therein is a twelfth embodiment 10k of the present soft-tissue fasteners. Fastener 10k is substantially similar to 10a of FIGS. 6A-6F, with the primary difference being that each of shafts 14a, 14b includes barbs 26d, 26e, 26f (not shown but 180° opposite barb 26d) like those of fastener 10d of FIGS. 15A-15B, and hollowed portions 138 like those of fastener 10f of FIGS. 17A-17C corresponding to barbs 26d, instead of three barbs 26b. In other embodiments, fastener 10k can include hollowed portions 138 for each of barbs 26e and/or 26f (additional or alternative to the depicted hollowed portions for each of barbs 26d).

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A soft-tissue fastener for coupling an implant to soft tissue, the soft-tissue fastener comprising:
an elongated shaft having a shaft length extending from a shaft proximal end to a shaft distal end, and an internal surface defining an internal passage extending along the shaft length;
an enlarged head coupled to the proximal end of the shaft, the enlarged head having a proximal side and a distal side;
a plurality of outriggers each extending from the enlarged head in a direction away from the internal passage to an outrigger distal end; and
a plurality of barbs each extending along a portion of the shaft length, each of the barbs having a leading edge and trailing edge spaced from the shaft proximal end, the leading edge of each barb being disposed between the trailing edge of that barb and the shaft distal end, where the trailing edge of a second one of the barbs is closer to the shaft proximal end than is the trailing edge of a first one of the barbs;
where the shaft, head, outriggers, and barbs are defined by a unitary piece of polymer.

2. The soft-tissue fastener of claim 1, where the polymer is bioresorbable.

3. The soft-tissue fastener of claim 1, where the trailing edge of a third one of the barbs is closer to the shaft proximal end than is the trailing edge of the second one of the barbs.

4. The soft-tissue fastener of claim 1, where the plurality of barbs are disposed at equiangular intervals around the cross-sectional perimeter of the shaft.

5. The soft-tissue fastener of claim 4, where the plurality of barbs consists of three barbs.

6. The soft-tissue fastener of claim 3, where at least one of the barbs is not radially aligned with any of the outriggers.

7. (canceled)

8. The soft-tissue fastener of claim 3, where:
at least a portion of the leading edge of the first one of the barbs is disposed at first barb angle relative to the central, longitudinal axis of the shaft;
at least a portion of the leading edge of the second one of the barbs is disposed at a second barb angle relative to the central, longitudinal axis of the shaft, and the second barb angle is smaller than the first barb angle; and
at least a portion of the leading edge of the third one of the barbs is disposed at a third barb angle relative to the central, longitudinal axis of the shaft, and the third barb angle is smaller than the second barb angle.

9-10. (canceled)

11. The soft-tissue fastener of claim 3, where:
an outer surface of the shaft defines a tapered section having an first outer transverse dimension at the shaft distal end and tapering toward the shaft proximal end to a second outer transverse dimension that is larger than the first outer transverse dimension, and
the tapered section of the shaft tapers at a first taper angle relative to a central, longitudinal axis of the shaft, and the first taper angle is substantially equal to the first barb angle.

12. (canceled)

13. The soft-tissue fastener of claim 11, where the inner surface and outer surface define an edge at the shaft distal end.

14. The soft-tissue fastener of claim 1, where each of the outrigger distal ends is, when measured parallel to a central, longitudinal axis of the shaft, closer to the shaft distal end and is at least a portion of the distal side of the head of the shaft.

15. The soft-tissue fastener of claim 1, where the plurality of outriggers are disposed at equiangular intervals around the head.

16. The soft-tissue fastener of claim 15, where the plurality of outriggers consists of four outriggers.

17. (canceled)

18. The soft-tissue fastener of claim 1, where the internal passage has a first inner transverse dimension at the shaft proximal end, a second inner transverse dimension at the shaft distal end, and the second inner transverse dimension is smaller than the first inner transverse dimension.

19-80. (canceled)

81. A bone fastener for coupling an implant to bone, the bone fastener comprising:
a first elongated shaft having a first shaft length extending from a first shaft proximal end to a first shaft distal end, and defining an internal first passage extending along the first shaft length;
a second elongated shaft having a second shaft length extending from a second shaft proximal end to a second shaft distal end, and defining an internal second passage extending along the second shaft length;
an enlarged head coupled to the proximal ends of the first shaft and the second shaft;
a plurality of first barbs each extending along a portion of the first shaft length, each first barb having a leading edge and trailing edge spaced from the proximal end of the first shaft, the leading edge of each first barb being disposed between the trailing edge of that first barb and the first shaft distal end;
a plurality of second barbs each extending along a portion of the first shaft length, each of the second barbs having a leading edge and a trailing edge disposed between the head and the trailing edges of the first barbs, the leading edge of each second barb being disposed between the trailing edge of that second barb and the first shaft distal end;
a plurality of third barbs each extending along a portion of the second shaft length, each third barb having a leading edge and trailing edge spaced from the proximal end of the second shaft, the leading edge of each third barb being disposed between the trailing edge of that third barb and the second shaft distal end; and
a plurality of fourth barbs each extending along a portion of the second shaft length, each of the fourth barbs having a leading edge and a trailing edge disposed between the head and the trailing edges of the third barbs, the leading edge of each fourth barb being disposed between the trailing edge of that fourth barb and the second shaft distal end;
where the shafts, head, and barbs are defined by a unitary piece of polymer.

82. The bone fastener of claim 81, where the polymer is not bioresorbable.

83. (canceled)

84. The bone fastener of claim 81, where the plurality of first barbs consists of four barbs, and the plurality of second barbs consists of four barbs.

85-86. (canceled)

87. The bone fastener of claim 81, where at least one of the second barbs is not radially aligned with any of the first barbs, and where at least one of the fourth barbs is not radially aligned with any of the third barbs.

88-97. (canceled)

98. The bone fastener of claim 81, where:
an outer surface of the first shaft defines a tapered section having an first outer transverse dimension at the first shaft distal end and tapering toward the first shaft proximal end to a second outer transverse dimension that is larger than the first outer transverse dimension; and
the tapered section of the first shaft tapers at a first taper angle relative to a central, longitudinal axis of the first shaft, and the first taper angle is substantially equal to the first barb angle.

99-103. (canceled)

104. The bone fastener of claim 98, where:
an outer surface of the second shaft defines a tapered section having an first outer transverse dimension at the second shaft distal end and tapering toward the second shaft proximal end to a second outer transverse dimension that is larger than the first outer transverse dimension; and
the tapered section of the second shaft tapers at a second taper angle relative to a central, longitudinal axis of the second shaft, and the second taper angle is substantially equal to the third barb angle.

105. (canceled)

106. The bone fastener of claim 14, where:
the inner surface and outer surface of the first shaft define an edge at the first shaft distal end; and
the inner surface and outer surface of the second shaft define an edge at the second shaft distal end.

107. The bone fastener of claim 81, where the first and second internal passages each has a first inner transverse dimension at the respective shaft proximal end, a second inner transverse dimension at the respective shaft distal end, and each second inner transverse dimension is smaller than the corresponding first inner transverse dimension.

108-124. (canceled)
